(19) United States
(12) Patent Application Publication
(10) Pub. No.: US 2009/0155782 A1
(43) Pub. Date: Jun. 18, 2009

(54) Homoeologous Region Determining Method by Homo Junction Fingerprint Method, Homoeologous Region Determining Device, and Gene Screening Method

(75) Inventor: Koichi Hagiwara, Saitama (JP)

Correspondence Address: DAY PITNEY LLP, 7 TIMES SQUARE, NEW YORK, NY, 10036-7311, US

(73) Assignees: TOMY DIGITAL BIOLOGY CO., LTD., Tokyo (JP); SAITAMA MEDICAL UNIVERSITY, Saitama (JP)

(21) Appl. No.: 11/988,812

(22) PCT Filed: Jul. 7, 2006
(86) PCT No.: PCT/JP2006/313616

§ 371 Date: Apr. 18, 2008

(30) Foreign Application Priority Data
Jul. 12, 2005 (JP) 2005-203654

(51) Int. Cl. C12Q 1/68; C12M 1/34

(52) U.S. Cl. 435/6; 435/287.2

(57) ABSTRACT

To provide a method for efficiently searching for a recessive disease gene without needing any pedigree analysis. In a homoeologous region determining method, the following steps are conducted. It is determined whether or not the base constituting a polymorphic marker of a sample DNA of diploid or higher polyploidy is a homojunction. Homojunction region information representing the region of the sample DNA where polymorphic markers determined as continuous homojunctions acquired. If the continuous probability and/or continuous distance of the polymorphic markers contained in the homojunction region information satisfy a predetermined determination condition, the homojunction region is determined as a homoeologous region. A homoeologous region determining device and a gene screening method for identifying a disease susceptibility gene from the determined homoeologous region are also provided.

### BACKGROUND OF THE INVENTION

1. Field of the Invention

The present invention relates to a method for efficiently searching for the locations of disease susceptibility genes for monogenic diseases or polygenic diseases caused by recessive genes using polymorphic markers.

2. Description of the Related Art

The identification of disease susceptibility genes for diseases caused by recessive genes is remarkably important for the development of disease treatment. An enormous amount of research related to such identification has been conducted for some time. Analysis methods have been developed for this purpose, such as methods that involve linkage analysis as well as affected sib-pair analysis and specifies disease susceptibility gene regions.

“Linkage analysis” refers to a method used to narrow down the location of a causal gene on a chromosome based on the degree of linkage that exists between a phenotype-related locus and a marker locus on the chromosome. Additionally, “affected sib-pair analysis” refers to a method used to narrow down the location of a causal gene by conducting a comparison among siblings with the same disease. A polymorphic marker is used for such analyses (refer to non-patent document 1). “Polymorphism” refers to a difference in DNA bases. It is defined with reference to variations of certain bases that occur in more than 1% of the population. However, in reality, variations of bases occurring in less than 1% of the population correspond to “polymorphisms” in some cases. In the present invention, all bases that have variations are considered polymorphic. “Polymorphic marker” refers to a polymorphism that is used as an indicator when disease susceptibility genes are searched for a specific DNA polymorphism. Regarding polymorphic markers, microsatellite polymorphisms, VNTR (Variable Number of Tandem Repeats) polymorphisms, and SNPs (Single Nucleotide Polymorphisms) are used for analysis. Polymorphism databases have been publicized, and such databases are used for analysis of disease susceptibility genes (refer to non-patent document 2). The dbSNP database (http://www.ncbi.nlm.nih.gov/SNP/index.html) and the JSNP (SNP for the Japanese people) database disclosed jointly by the Japan Science and Technology Corporation and the Institute of Medical Science of the University of Tokyo (http://snp.ims.u-tokyo.ac.jp) and the like are examples of such databases.

Additionally, as an identification method for recessive disease genes, a homozygosity mapping method that uses polymorphisms and the like is known. One method uses restriction fragment length polymorphisms (RFLP), which are SNPs (refer to non-patent document 3). Another method uses microsatellite polymorphisms (refer to non-patent document 4).

Furthermore, there exists a type of analysis known as associated analysis that is a well-known method for identifying a disease susceptibility gene region. The associated analysis involves comparing the frequency of appearance of specific polymorphic markers in a control group and a diseased group, through which the locations of causal genes are narrowed down. SNP is used for this method.

As an example of disease susceptibility gene identification that has actually been conducted by the linkage analysis and/or the associated analysis method mentioned above, the identification of a causal gene for type II diabetes (refer to non-patent document 1) is well known.

[Patent document 1] Patent application 2002-339901

[Non-patent document 1] “Genomuigaku Kara Genomuiryo He (Genome medicine to genome medical care)” written by Yusuke Nakamura in 2005

[Non-patent document 2] Sellick, G. S. et al. Diabetes 52:2636, 2003

[Non-patent document 3] Lander, E. S. et al. Science 236:1567, 1987

[Non-patent document 4] Kobayashi, K. et al. Nature Genetics 22:159, 1997

[Non-patent document 5] “An Introduction to Population Genetics Theory,” 8^{th} version, written by Crow, J. F. and translated by Kimura Motoo (1991) (BAIFUKAN CO., LTD, Publishing Company)

[Non-patent document 6] Mariotta, S. et al. Sarcoidosis Vasc. Diffuse Lung Dis. 21:173-81, 2004

[Non-patent document 7] Castellana G. & Lamorgese V., Respiration 70:549-55, 2003

[Non-patent document 8] Tachibana T. et al. Sarcoidosis Vasc. Diffuse Lung Dis. 18(suppl 1), 58, 2001

[Non-patent document9] Huqun. et al. Submitted.

### SUMMARY OF THE INVENTION

### Problems to Be Solved by the Invention

Linkage analysis and affected sib-pair analysis are based on pedigree analysis. The aforementioned types of analysis involve difficulties in processes used to obtain samples as a step prior to performance of gene analysis thereof. In particular, in relation to low-permeability diseases, in many cases, preservation of the number of samples that can lead to a significant conclusion constituting a rate-determining step for analyses. Associated analysis has disadvantages in that such analysis requires a control group and retesting must be conducted due to the occurrence of many false-positive results. Furthermore, in regards to a disease susceptibility gene that uses a polymorphic marker, based on the concept of conventional linkage and linkage disequilibrium, horizontal linkage among polymorphic markers has been focused. Thus, there has existed a problem in which many samples were required and enormous costs and time were incurred.

### Means of Solving the Problems

In regards to recessive diseases, there are some cases in which a homologous gene deriving from a single gene of a single ancestor gives rise to a state of homozygosity, thus causing a disease. The present inventor discovered that all base sequences corresponding to polymorphisms, such as for genetic abnormalities, SNPs, and microsatellite polymorphisms within regions in which a disease susceptibility gene exists correspond to a state of homozygosity. Based on this fact, disease susceptibility genes exist within regions in which homozygous polymorphic markers are contiguous. That is to say, it is highly possible that a region in which polymorphic markers are contiguous and indicate homozygosity in regards to a recessive gene is a homologous region. According to the present invention, based on such discovery, a homologous region determining method that can result in a determination based on a small number of samples with the use of polymorphic markers is provided. Additionally, in the present invention, a homologous region determining device that determines whether a relevant region is a homologous region or not using polymorphic markers is provided. Moreover, a gene screening method for searching for a disease gene within the regions determined by the homologous region determining method or homologous region determining device is provided. That is to say, the present invention is as follows.

(1) The present invention provides a homologous region determining method, comprising the steps of determining whether the bases making up polymorphic markers of sample DNA indicating a state of diploidy or polyploidy indicate homozygosity, acquiring the homozygous region information showing the region of sample DNA in which the polymorphic markers that have been determined as corresponding to a state of homozygosity are contiguous, from among the polymorphic markers that have become the subject of the determination by the homozygosity determining step, and determining that a homozygous region is a homologous region, when continuous probability and/or continuous distance regarding polymorphic markers included in the homozygous region information satisfy given homologous determination conditions.

(2) The present invention provides a homologous region determining method, comprising the steps of selecting polymorphic markers as the subject of determination regarding homozygosity selected from among polymorphic markers of sample DNA indicating a state of diploidy or polyploidy, determining whether the bases making up the polymorphic markers selected by the polymorphic marker selection section indicate homozygosity or not, acquiring the homozygous region information showing the sample DNA region in which the polymorphic markers that have been determined as corresponding to a state of homozygosity by the homozygosity determining step are contiguous, and determining that a homozygous region is a homologous region, when continuous probability and/or continuous distance regarding polymorphic markers included in the homozygous region information satisfy given homologous determination conditions.

(3) The present invention provides the homologous region determining method, wherein the polymorphic marker selection step selects polymorphic markers through all chromosome regions of the sample DNA.

(4) The present invention provides the homologous region determining method, wherein the polymorphic marker selection step selects polymorphic markers included in regions corresponding to candidate gene regions.

(5) The present invention provides the homologous region determining method of any one of claims 1 through 4, wherein the sample DNA is of plant origin.

(6) The present invention provides the homologous region determining method of any one of claims 1 through 4, wherein the sample DNA is of animal origin.

(7) The present invention provides the homologous region determining method of any one of claims 1 through 4, wherein the sample DNA is of human origin.

(8) The present invention provides the homologous region determining method of any one of claims 1 through 4, wherein the sample DNA is of Japanese origin.

(9) The present invention provides the homologous region determining method of any one of claims 1 through 8, wherein the polymorphic markers correspond to SNPs.

(10) The present invention provides the homologous region determining method of any one of claims 1 through 8, wherein the polymorphic markers correspond to microsatellite polymorphism.

(11) The present invention provides the homologous region determining method, wherein the polymorphic markers correspond to VNTR polymorphism.

(12) The present invention provides the homologous region determining method, wherein polymorphic markers are based on a combination of more than two of any of SNP, microsatellite polymorphism, or VNTR polymorphism.

(13) The present invention provides the homologous region determining method as the step in which the sample DNA is of human origin and in which 10,000 or more SNPs from all chromosome regions of the sample DNA are selected.

(14) The present invention provides the homologous region determining method as the step wherein the sample DNA is of human origin and which selects 100,000 or more SNPs in all chromosome regions of the sample DNA

(15) The present invention provides the homologous region determining method, wherein in regards to homologous determining conditions of the homologous region determining step, the probability of a homozygous region repeating regarding the polymorphic markers shown in the homozygous region information is a value smaller than the probability that selected from a range of 1/10,000,000 to 1/10,000.

(16) The present invention provides the homologous region determining method, wherein in regards to homologous determining conditions of the homologous region determining step, the probability of a homozygous region repeating regarding the polymorphic markers shown in the homozygous region information is a value smaller than the probability that selected from a range of 1/5,000,000 to 1/50,000.

(17) The present invention provides the homologous region determining method, wherein in regards to homologous determining conditions of the homologous region determining step, the probability of a homozygous region repeating regarding the polymorphic markers shown in the homozygous region information is a value smaller than the probability that selected from a range of 1/1,000,000 to 1/100,000.

(18) The present invention provides the homologous region determining method, wherein in regards to homologous determining conditions of the homologous region determining step, the probability of a homozygous region repeating regarding the polymorphic markers shown in the homozygous region information is a value smaller than the probability that selected from a range of 1/1,000,000 to 1/5,000.

(19) The present invention provides the homologous region determining method, further comprising the steps of acquiring the homologous region information showing a region that has been determined as being a homologous region by the homologous region step in response to multiple samples, and of acquiring the frequency of occurrence of overlapping specific homologous regions among multiple samples obtained based on the homologous region information for multiple samples that has been acquired by the homologous region information acquisition step.

(20) The present invention provides a gene screening method in which genetic sequences included in the homologous regions determined by the homologous region determining methods mentioned in any one of the above descriptions are identified and are compared with sequences of normal genes.

(21) The present invention provides a gene screening method in which whether or not the homologous regions determined by the homologous region determining methods mentioned in any one of the above descriptions could contain genes that have already been known to function in a homozygous state is determined, and in the case of a region that could contain a gene that has been already known, sequences of corresponding known genes and corresponding genes of sample DNA are compared.

(22) The present invention provides a gene screening method in which in case that the sample DNA corresponds to a disease, in case that the homologous regions determined by the homologous region determining methods mentioned in any one of the above descriptions contain a gene that is expected to be related to a corresponding disease, the sequences of the corresponding genes in the homologous region of the sample DNA are identified and compared with normal genes.

(23) The present invention provides a homologous region determining device, comprising a homozygosity determining section in which whether or not bases comprising polymorphic markers in sample DNA indicating a state of diploidy or polyploidy indicate homozygosity is determined, a homozygous region information acquisition section in which from among polymorphic markers as the subject of determination carried out by the homozygosity determining section, polymorphic markers that have been determined as indicating homozygosity acquire bomozygous region information showing a sequential sample DNA region, and a homologous region determining section in which continuous probability and/or continuous distance regarding polymorphic markers included in homozygous region information that will be acquired by the homozygous region information acquisition section satisfy given homologous determination conditions, it is determined that a homozygous region is a homologous region.

(24) The present invention provides a homologous region determining device comprising, a polymorphic marker selection section in which polymorphic markers as the subject of determination regarding homozygosity are selected from among polymorphic markers of sample DNA indicating a state of diploidy or polyploidy, a homozygosity determining section in which whether the bases making up the polymorphic markers selected by the polymorphic marker selection section indicate homozygosity or not is determined, a homozygous region information acquisition section in which from among polymorphic markers as the subject of determination carried out by the homozygosity determining section, polymorphic markers that have been determined as indicating homozygosity acquire homozygous region information showing a sequential sample DNA region, and a homologous region determining section in which when continuous probability and/or continuous distance regarding polymorphic markers included in homozygous region information that will be acquired by the homozygous region information acquisition section satisfy given homologous determination conditions, it is determined that a homozygous region is a homologous region.

(25) The present invention provides the homologous region determining device, wherein polymorphic markers are selected through all chromosome regions of the sample DNA.

(26) The present invention provides the homologous region determining device, wherein the polymorphic marker selection step selects polymorphic markers included in regions corresponding to candidate gene regions at the polymorphic marker selection section.

(27) The present invention provides the homologous region determining device of, wherein the sample DNA is of plant origin.

(28) The present invention provides the homologous region determining device, wherein the sample DNA is of animal origin.

(29) The present invention provides the homologous region determining device, wherein the sample DNA is wherein the sample DNA is of human origin.

(30) The present invention provides the homologous region determining device of any one of claims 23 through 26, wherein the polymorphic markers correspond to SNPs.

(31) The present invention provides the homologous region determining device, wherein the polymorphic markers correspond to SNPs.

(32) The present invention provides the homologous region determining device, wherein the polymorphic markers correspond to microsatellite polymorphism.

(33) The present invention provides the homologous region determining device, wherein the polymorphic markers correspond to VNTR polymorphism.

(34) The present invention provides the homologous region determining device, wherein polymorphic markers are based on a combination of more than two of any of SNP, microsatellite polymorphism, or VNTR polymorphism.

(35) The present invention provides the homologous region determining device in which the sample DNA is of human origin and in which 10,000 or more SNPs from all chromosome regions of the sample DNA are selected at the polymorphic marker selection section.

(36) The present invention provides the homologous region determining device in which the sample DNA is of human origin and which selects 100,000 or more SNPs in all chromosome regions of the sample DNA at the polymorphic marker selection section.

(37) The present invention provides the homologous region determining device in which in regards to homologous determination conditions, the continuous probability of a homozygous region regarding the polymorphic markers shown in the homozygous region information can be a smaller value than that selected from a scope of 1/10,000,000 to 1/10,000 at the homologous region determining section.

(38) The present invention provides the homologous region determining device in which in regards to homologous determination conditions, the continuous probability of a homozygous region regarding the polymorphic markers shown in the homozygous region information can be a smaller value than that selected from a scope of 1/5,000,000 to 1/50,000 at the homologous region determining section.

(39) The present invention provides the homologous region determining device o in which in regards to homologous determination conditions, the continuous probability of a homozygous region regarding the polymorphic markers show in the homozygous region information can be a smaller value than that selected from a scope of 1/1,000,000 to 1/100,000 at the homologous region determining section.

(40) The present invention provides the homologous region determining device in which in regards to homologous determination conditions, the continuous probability of a homozygous region regarding the polymorphic markers show in the homozygous region information can be a smaller value than that selected from a scope of 1/1,000,000 to 1/5,000 at the homologous region determining section.

(41) The present invention provides the homologous region determining device in which the homologous region information as information showing the homozygous region determined to satisfy the homologous determination conditions by the homologous region determining section is visualized and outputted at the homologous region determining section.

(42) The present invention provides the homologous region determining device, further comprising a homologous region information preservation section in which multiple pieces of the homologous region information showing a region that has been determined as being a homologous region by the homologous region determining section are preserved in response to multiple samples; and a homologous region overlapping frequency information acquisition section in which the homologous region overlapping frequency information showing the overlapping frequency among multiple samples in regards to specific homologous regions is acquired based on homologous region information for multiple samples preserved by the homologous region information preservation section.

(43) The present invention provides the homologous region determining device, further comprising a homologous region overlapping frequency visualization information output section in which the homologous region overlapping frequency visualization information corresponds to visualized and outputted homologous region overlapping frequency information obtained by the homologous region overlapping frequency information acquisition section.

(44) The present invention provides the homologous region determining device of claim 42 or 43, further comprising, a homologous region information accumulation section in which the overlapping frequency obtained through the homologous region overlapping frequency acquisition section is adjusted to the homologous region information, and that the resulting information is accumulated, and an important homologous region information acquisition section in which from among the homologous region information accumulated in the homologous region information accumulation section, the homologous region information associated with a frequency that is greater than or equal to a given overlapping frequency is acquired.

(45) The present invention provides the homologous region determining device, further comprising a homologous region information output section in which the homologous region information to which more than or equal to given overlapping frequency is adjusted and such information is obtained by the important homologous region information acquisition section is visualized and outputted.

(46) The present invention provides a gene screening method in which genetic sequences included in the homologous regions determined by the homologous region determining devices mentioned in any one of the above descriptions are identified and are compared with sequences of normal genes

(47) The present invention provides a gene screening method in which the homologous regions identified by the homologous region determining devices mentioned in any one of the above descriptions are overlapped with the homologous region for which information is accumulated in the homologous region information accumulation section, and the gene sequences included in the overlapping region are identified and compared with the sequences of normal genes.

(48) The present invention provides a gene screening method in which it is determined whether or not the homologous regions determined by the homologous region determining devices mentioned in any one of the above descriptions could contain genes that have already been known to function in a homozygous state, and in the case of a region that could contain a gene that has been already known, sequences of corresponding known genes and corresponding genes of sample DNA are compared.

(49) The present invention provides a gene screening method in which in case that the sample DNA corresponds to a disease, if the homologous regions determined by the homologous region determining devices mentioned in any one of the above descriptions contain a gene that is expected to be related to a corresponding disease, the sequences of the corresponding genes in the homologous region of the sample DNA are identified and compared with normal genes.

### ADVANTAGEOUS EFFECT OF THE INVENTION

The new determining method that recognizes a homologous region based on population genetics according to the present invention does not require pedigree analysis or a control group when searching for a disease susceptibility gene related to a human recessive gene. Therefore, it is easy to preserve samples and possible to remarkably reduce the number of analyses carried out. Also, even in cases in which diseases are not currently occurring, it can be said that homologous regions are vulnerable portions in relation to diseases. This matter is also useful from the viewpoint of preventive medicine. Moreover, by applying the present invention to plants and animals, it is possible to search for a causal gene in the same manner as with a human being in relation to recessive gene diseases. Also, it is possible to discover genes that carry out useful functions in terms of homozygosity and useful phenotype-related genes.

### DETAILED DESCRIPTION OF THE PREFERRED EMBODIMENTS

Hereinafter, the preferred embodiments for carrying out the present inventions are explained. The present inventions are not limited to such preferred embodiments, and can be implemented in various forms without deviation from the spirit or the main characteristics thereof.

Prior to providing explanations related to the present invention, the concept of inbreeding coefficient as it pertains to population genetics as a prerequisite of the present invention is explained hereinafter. Inbreeding enhances homozygous characteristics and the frequency of occurance of recessive gene diseases. It is possible to think that the genetic influence of inbreeding is based on homologous genes. “Homologous” refers the sharing of a common ancestor, and “homologous genes” are genes in a single individual derived from a single chromosome of a single ancestor. “Inbreeding coefficient” refers to the percentage of the total number of genes accounted for by homologous genes (non patent document 5). Similarly, a homologous chromosome region is defined, and the ratio of the homologous chromosome region to the totality of chromosome regions constitutes the inbreeding coefficient. In the present invention, a homologous chromosome region is called a “homologous region.”

FIG. 1 is a figure, which explains the concept of homologous region. A child inherits a single chromosome from the father and a single chromosome from the mother. Thus, the relatedness to a given ancestor decreases by ½ every generation, and the lengths of homologous regions become shorter as well. Additionally, due to crossover, which takes place at the time of meiosis, variations occur. B and C inherit ½ of A's chromosomes, and D and E inherit ¼ of A's chromosomes. In a case in which parents (D and E) are involved in a cross-cousin marriage, the inbreeding coefficient for the child (F) becomes 1/16. In such a case, it may be possible to receive a gene from the same ancestor from both the father and mother, such as in the case of F. Such regions that have become homozygous are homologous regions. In case that a gene related to a recessive gene disease exists within a homologous region, disease will occur. This is because abnormalities that are normally shrouded in normal alleles have emerged. This is a reason why diseases tend to occur easily in consanguineous marriages.

However, there are some cases in which a disease that is deemed to be recessive gene disease afflicts a family without the occurrence of any consanguineous marriages. Such cases do not contradict the concept of inbreeding coefficient simply as a result of the lack of consanguineous marriages. This is because there is a possibility that a given homologous region stems from not only close ancestors such as grandparents and great-grandparents, and the like, but also from ancestors in the distant past. Homologous regions become shorter due to crossover as generations pass. Thus, probability falls and relevant diseases are unlikely to occur. Additionally, mutation may be a possible reason why an affected gene would become homozygous. However, the probability thereof is thought to be 1/106-1/105 per gene per generation. Thus, such matter is not considered to be relevant to the present invention.

According to the concept of inbreeding coefficient mentioned above, the items that have become homozygous within a homologous region must be all genes and polymorphic base sequences within a homologous region as well as disease susceptibility genes. In contrast, a region in which polymorphisms are contiguous and indicate homozygosity has a high possibility of being a homologous region. Also, in such case, there is a high possibility that disease susceptibility genes for diseases caused by recessive genes exist. Such matter is explained by using FIG. 4. FIG. 4 shows a case where a polymorphic marker is SNP, and bold-letter base is SNP. As shown in FIG. 1, chromosomes are normally passed down via two routes: from a father derivation and from a mother derivation. Thus, polymorphism portions mix homozygous and heterozygous regions (heterojunction applies to all SNPs in FIG. 4). However, in a homologous region, all base sequences correspond to a state of homozygosity as per region A. Thus, polymorphisms can be used as markers, and it is highly likely that a homozygous region in which homozygous polymorphic markers are contiguous would be a homologous region.

The present invention provides a homologous region determining method and device using polymorphisms as markers based on the concepts mentioned above. The homologous region determining method according to the present invention is called the “homozygosity fingerprinting method.” Furthermore, the present invention also offers a gene screening method that uses the homozygosity fingerprinting method.

A first embodiment mainly relates to claims 1, 5 through 12, 15 through 18, 23, 27 through 34, and 37 through 40. A second embodiment mainly relates to claims 2 through 4, 13, 14, 24 through 26, and 35, and 36. A third embodiment mainly relates to claims 19 and 42. A fourth embodiment mainly relates to claims 44. A fifth embodiment mainly relates to claims 41. A sixth embodiment mainly relates to claims 43 and 45. A seventh embodiment mainly relates to claims 20 and 46. An eighth embodiment mainly relates to claims 21 and 48. A ninth embodiment mainly relates to claims 22 and 49. A tenth embodiment mainly relates to claim 47.

### First Embodiment

### Structure of a First Embodiment

A first embodiment is explained hereinafter. An example of a functional block of the embodiment is shown in FIG. 2. The homologous region determining device of the embodiment (0200) comprises the homozygosity determining section (0201), the homozygous region information acquisition section (0202), and the homologous region determining section (0203).

The homozygosity determining section (0201) is configured so as to determine whether or not bases comprising polymorphic markers in sample DNA indicating a state of diploidy or polyploidy indicate homozygosity. As a polymorphism typing method, the PCR-SSCP, PCR-RFLP, direct sequencing method, MALDI-TOF/MS method, TaqMan method, invader method, and the like can be used. The homozygosity determining section (0201) determines whether bases for which typing has been conducted via the aforementioned methods indicate homozygosity or not.

“Sample DNA” is genome DNA that serves as a sample used for identifying polymorphisms. Such sample DNA is not particularly limited, as long as such sample contains DNA indicating a state of diploidy or polyploidy. Samples may be of human origin, of non-human animal origin, and furthermore, of plant origin. In the case of samples of human origin, samples taken from a human of Japanese origin are desirable. The reason why the Japanese-derived DNA is desirable is that Japan is an insular country, which undertook a policy of isolationism. Due thereto, interbreeding with members of other ethnicity was less common, and thus Japan exhibits the phenomenon of high inbreeding coefficients. There is a high probability that a Japanese individual would exhibit a homologous region. On the other hand, for example, the U.S. is a country in which interbreeding among races takes place frequently, and it exhibits the phenomenon of low inbreeding coefficients. Due to crossover, homologous regions are shorter. Thus, there is a low probability of homologous states occurring. Additionally, it becomes difficult to determine whether a sequence indicating homozygosity is a result of a coincidence or is due to a homologous state. Samples that allow use of genome DNA, such as blood, saliva, tissue, or cells, are acceptable. The reason why DNA indicating a state of diploidy or polyploidy applies is that whether or not a homologous chromosome indicates homozygosity cannot be determined based on a condition of monoploidy in the present invention. Therefore, in regards to sex chromosomes, in the case of females, an X chromosome can be in a homozygous state. Thus, it is possible to make relevant determinations. However, detection is impossible for males. Additionally, DNA indicating a state of triploidy or polyploidy is acceptable. The method of preparing genome DNA is not particularly limited, as long as a method suitable for the polymorphism typing method is used. For instance, when a method for conducting PCR is used, genome DNA must be prepared so that substances that are PCR inhibitors (EDTA, and the like) are not present.

A “polymorphic marker” uses a polymorphism, which involves a difference in DNA bases, as a marker when a disease susceptibility gene is searched for. Examples of polymorphisms include microsatellite polymorphisms, VNTR polymorphisms, and SNPs. As mentioned above, various polymorphism databases have been publicized. Tandem repeats of from two to dozens of bases exist on DNA. Most thereof do not have genetic information and exist in functionally unknown portions, and differences tend to take place among individual organisms. The frequency of occurrence of such repeated portions differs from individual to individual, and corresponds to polymorphism. Among such polymorphisms, polymorphisms of several to dozens of bases are called “VTR polymorphisms.” And polymorphisms of two to four bases are called “microsatellite polymorphisms.” Additionally, “SNP” refers to a type of polymorphism that depends on monobasic differences in DNA. RFLP is contained in SNP. It is said that SNP frequently can be found in base sequences. It is also said that there is about one SNP per 300 bases in human beings, and 3 million to 10 million SNPs exist among the totality of chromosomes. In recent years, searches for disease susceptibility genes have been undertaken using such SNP differences. In the present invention, a microsatellite polymorphism or a VNTR polymorphism can be used as a polymorphic marker. Due to the existence of many polymorphisms, it is desirable to use SNP as a polymorphic marker in the present invention. Furthermore, a combination of more than two of any of SNP, microsatellite polymorphism, or VNTR polymorphism is acceptable.

“Homozygosity” refers to a situation in which homologous chromosomes have the same bases. That is to say, both of the opposing bases derived from the father and from the mother (pair of opposing bases) are the same. And a homozygous base pair corresponds to a state of homozygosity. A homozygous state does not involve a chromosome indicating a state of diploidy, and may be one indicating a state of triploidy or polyploidy. In such case, in case that all chromosomes that become pairs have the same bases, such bases can be said to indicate homozygosity.

The homozygous region information acquisition section (0202) is configured so that from among polymorphic markers as the subject of determination carried out by the homozygosity determining section (0201) mentioned above, polymorphic markers that have been determined as indicating homozygosity acquire homozygous region information showing a sequential sample DNA region. “Contiguous” refers to a situation in which polymorphic markers that have been determined as corresponding to a state of homozygosity by the homozygosity determining section mentioned above line up without pinching heterojunction polymorphic markers. Additionally, “homozygous region” refers to a region of sample DNA where polymorphic markers determined as corresponding to homozygosity are contiguous. “sequential sample DNA region” refers to a DNA region pinched between polymorphic markers including genes as well as polymorphic markers. Explanations are given by with reference to FIG. 3A. First of all, 3A shows that polymorphic markers exist on DNA (0301). Black bars indicate homozygosity polymorphic markers, and white bars indicate heterozygote polymorphic markers. The portion shown as 0302 indicates that all polymorphic markers (b, c, d, and e) indicate homozygosity. Thus, such region is sequential. Therefore, the shaded DNA region in FIG. 3 is a homozygous region. “Homozygous region information” refers to information corresponding to sequential sample DNA. For instance, in the case of FIG. 3A, such information corresponds to information such as the location and ID of polymorphic markers (b, c, d, and e) included in a homozygous region, and sequential regions for of b through e.

The homologous region determining section (0203) is configured so that when continuous probability and/or continuous distance regarding polymorphic markers included in homozygous region information that will be acquired by the homozygous region information acquisition section (0202) satisfy given homologous determination conditions, it is determined that a homozygous region is a homologous region. “Continuous probability” refers to the probability of polymorphic markers being contiguous and indicating homozygosity. In regards to polymorphisms, the probability of such polymorphisms indicating homozygosity (homozygosity ratio) has been computed.

The probability differs from group to group. Thus, it would be better to use probability that is suitable for a given sample. For example, in the case of human beings, the homozygosity ratio concerning polymorphisms differs between the Japanese group and the American group. Thus, in the case of Japanese samples, it is desirable to compute portability using the homozygosity ratio for Japanese or for Asians. Computation is acceptable by using targeted samples for each group regarding which detection is undertaken. “Continuous probability” is the value resulting when the homozygosity ratio for continuous polymorphic markers is multiplied, and it represents the probability of a sequence indicating a homozygous state as a result of a coincidence. “Continuous distance” refers to the length of a polymorphic marker contiguous that indicates homozygosity. “Length” refers to physical (map) length, using the unit of the base pair. That is to say, “continuous distance” refers to the length between the polymorphic markers of both ends of a homozygous region. “Homologous determination conditions” refer to conditions concerning continuous probability or continuous distance that are determination standards regarding whether a sequence indicating homozygosity corresponds to a homologous region or not. Polymorphic markers indicate either homozygosity or heterojunction. Thus, there could be a possibility of a sequence indicating a homozygous state as a result of a coincidence. In order to exclude regions in which sequences result from coincidences, relevant conditions are established. For instance, a homozygous region in which the continuous probability becomes less than or equal to 1/105 can be established as a homologous region. The probability shows that when determination is made using 105 polymorphic markers, only about one portion is determined as a homologous region that results from the coincidental existence of homozygosity. Additionally, the homologous determination conditions can be determined by continuous distance. A relevant continuous distance can be also determined by the average homozygosity ratio value concerning polymorphic marker to be detected and average value of the length between polymorphic markers. For example, when polymorphic markers of 100,000 locations are detected, the average value of the homozygosity ratio thereof is 0.74, and an average value between polymorphic markers of 23.6 kb, 900 kb, or more can be established as a homologous determination condition. When the ratio is unknown, the continuous probability of existence of homozygosity cannot be known. Thus, it is desirable to use the continuous distance that can be obtained from the average value of the homozygosity ratio as a homologous determination condition. Alternatively, both continuous probability and continuous distance may be used. The homologous region determining section (0203) recognizes a homozygous region that satisfies the homologous determination conditions as a homologous region.

However, in case that a homologous region is a region between polymorphic markers that have been recognized as indicating homozygosity, there is a possibility that a region that exists up to the polymorphic marker that has been determined to be a heterojunction region adjacent to the homozygosity polymorphic marker at both ends of the aforementioned markers may be determined as being not homologous, despite the fact that the aforementioned region is homologous. Thus, the portion up to the polymorphic marker that has been determined as being heterojunction adjacent to the homozygosity polymorphic meeting the homologous determination conditions may be included in a homologous region.

In regards to homologous determination conditions, the continuous probability of a homozygous region being a significant homologous region can be less than or equal to 1/10⁷-1/10⁴. Due to the number of polymorphic markers, in the case of probability that is greater than or equal to 1/10⁴, there is a possibility that there would tend to exist many continuous homozygous regions that would be determined as being homologous regions. And in the case of probability that is less than or equal to 1/10⁷, if the inbreeding coefficient is low, there is a possibility that the number of regions that would be recognized as homologous regions would be too small. It is said that human SNP is 107 units. Thus, when all SNPs are detected and there exists a portion in which a homozygous sequence is coincidental and is less than or equal to one portion, such region can be said to be a significant homologous region. Preferably, in relation to homologous determination conditions, continuous probability can be less than or equal to 1/(5×10⁶)-1/(5×10⁴). Further preferably, in relation to homologous determination conditions, the continuous probability can be less than or equal to 1/10⁶-1/10⁵. In case that the number of polymorphic markers is small, in relation to homologous determination conditions, the continuous probability can be less than or equal to 1/10⁶-1/(5×10³).

As a homologous region undergoes generations, such region becomes shorter due to crossover, and has diversities. Due to this fact, it can be said that a homologous region is like a fingerprint, which differs from individual to individual. Thus, the present inventor has called a homologous region determining method the “homozygosity fingerprinting method.”

Here, the probability that a region determined as being a homologous region could turn out not to be homologous is considered. In a hypothetical case in which chromosomes have infinite length, “1 cM (centiMorgan)=1 Mb (megabase)” and crossover randomly takes place on the chromosomes at the time of meiosis, the length of the fragments becomes exponentially distributed. M (Morgan) is a unit representing a genetic length as an expected value of crossover frequency taking place between 2 locations. 1 M is defined as a length at which one crossover can be expected per instance of meiosis. “1 cM= 1/100M.” In a case in which a father and mother of a patient have common ancestors, a homologous region is a common portion of a chromosome fragment of common ancestors inherited from each parent. The length of homologous region is exponentially-distributed. The probability density of the exponential distribution is indicated based on the following formula.

f(x)=λe^{-λx} [Mathematical formula 1]

Regarding a patient's chromosomes as chromosomes resulting after m instances of meiosis since the time of a given ancestor, the ancestor's chromosomes exist as a fragment with an average length of 100,000/mkb within the patient's chromosomes. A homologous region is a portion shared in common with an ancestor's chromosome fragments. Thus, in a case in which the frequency of occurrence of meiosis from the time of a given ancestor until the birth of patient is denoted with “m” for the side of the father and “n” for the side of the mother, the average length of a homologous region can be computed by 100,000/(m+n) kb. Therefore, the average fragment length is represented by the following formula.

$$\frac{1}{λ} = \frac{100000}{m + n} ( kb ) \qquad [ Mathematical formula 2 ]$$

In relation to a cross-cousin marriage, “m=m=3” applies. And in relation to a marriage between second cousins, “m=m=4” applies. Therefore, λ values for a child born from parents in a cross-cousin marriage and from a marriage of second cousins become 0.00006 and 0.00008, respectively. Here, since it is assumed that chromosomes hypothetically have infinite length at the beginning, and thus computation is simplified. However, the length of a homologous region is far shorter than that of the chromosomes, and due to such simplification, no major miscalculation would occur. In regards to homologous determination conditions, in case that a continuous distance is established as being 900 kb or more, despite the fact that a relevant region is homologous, the length of a homozygous region would be shorter than 900 kb. The probability that such case would not be a case involving a homozygous region can be computed based on the following formula.

$$\begin{matrix}P = {\int }_{0}^{900} {λe}^{- λ x} d x \\ = {[ - {e}^{- λ x} ]}_{0}^{900} \\ = - {e}^{- 900 λ} + 1\end{matrix} \qquad [ Mathematical formula 3 ]$$

The P values for a child born from parents of a cross-cousin marriage and a marriage of second cousins are 0.05 and 0.07, respectively. Thus, the probability of a homologous region actually being homologous would be 0.95 and 0.93, respectively. This shows that a homologous region can be determined with high probability. Similar to the case above, even when an ancestor from 20 generations before is commonly shared, there is a chance of about 70% that a homologous region can be detected. When it is intended to lower the probability that an actually homologous region would be excluded as being a non-homologous region, it is possible to establish homologous determination conditions in a loose manner.

One example of a computer-based configuration comprising a homozygosity determining section, a homozygosity information acquisition section, and a homologous region determining section as mentioned above is given as follows.

First of all, the homozygosity determining section acquires base sequence data for sample DNA indicating a state of diploidy or polyploidy for each chromosome. Such data is composed of location information, which specifies locations of the bases for each chromosome, and base type information, which specifies types of bases (adenine, guanine, cytosine, and thymine) related to the aforementioned location information. Such data is called “basic sample DNA data.” In regards to such basic sample DNA data, the output data of sequencer, and the like is acquired via communication and recording media, and the resulted data is stored in a storage area, such as a hard disk drive or RAM.

Additionally, the location information and homozygosity ratio information regarding a polymorphic marker are stored as a polymorphic marker file. Here, “homozygosity ratio information” refers to information concerning the probability that specific polymorphic markers would become homozygous, and such probability is generally acquired statistically. The location information regarding polymorphic markers is sequentially read from the storage region. And based on the read location information regarding polymorphic markers as a key, the process of searching for the aforementioned storage region is executed. The base type information to which such location information is related is acquired from basic sample DNA data of chromosomes, and the resulting information is temporarily stored in a storage region. Subsequently, it is determined whether or not the base type information stored temporarily in the storage region to which the same location information is related in regards to chromosomes is the same for all location information via the use of the comparison function of a CPU. In relation to location information for which comparison results are the same, a mark to the effect that such results are the same is made. And in the case that the results are not the same depending on relevant design, a mark to the effect that such results are not the same is made. And such information is stored in storage region as a file related to location information. Such file is called a “homozygosity location information file.”

Subsequently, from among the homozygosity location information files stored in the storage region, the homozygosity information acquisition section extracts information regarding continuous homozygous regions. Such “extraction” means that the location information relating to homozygosity is sequentially read out, and whether or not such location information corresponds to a positional relationship in which polymorphic markers are contiguous is determined. In case that the location information relating to homozygosity corresponds to a positional relationship in which polymorphic markers are contiguous, a sequential mark to such effect is recorded in relation to the aforementioned two pieces of location information. In case that the location information is related to a specific sequential mark, if such location information shares location information related to another sequential mark, the relevant sequence shows that three or more polymorphic markers are contiguous and are homozygous. A file in which such sequential marks and location information are related to each other is stored in the storage region as a sequential mark file.

Next, the homologous region determining section determines whether from among sequential mark files, sharing of the location information is contiguous or not, and determines whether a homozygous region corresponds to a homologous region or not according to the degree of such sequence. Specifically, based on such determination, the homozygosity ratio information stored as being related to the location information regarding sequent polymorphic markers is sequentially multiplied, and the probability that such sequence takes place due to reasons other than being homologous is computed. The computed probability is preserved in a given storage region once, and the values stored in other storage regions as homologous determination conditions are obtained. And comparison between the computed probability preserved in a given storage region and the values is executed using the comparison function of a CPU. As a result of comparison, in case that the computed probability is determined as being a smaller probability than that determined by homologous determination conditions, the location information showing corresponding regions is stored in the storage region as location information showing a homologous region. The location information indicating the homologous region contains all polymorphic marker information included in the homologous regions as well as the location information regarding polymorphic markers indicating both ends of the homologous region. Such file is called a “homologous region file.” Ultimately, when the location information stored in the homologous region file is outputted, it is possible to specify the homologous region.

### Description of a First Embodiment

FIG. 5 shows a description of processing concerning the homologous region determining method of the first embodiment. First of all, it is determined whether bases that are composed of polymorphic markers of sample DNA indicating a state of diploidy or polyploidy correspond to a state of homozygosity or not (homozygosity determining step: S0501). Subsequently, from among the polymorphic markers that have become the subject of the determination by the aforementioned homozygosity determining step, the homozygous region information showing the region of sample DNA in which the polymorphic markers that have been determined as corresponding to a state of homozygosity (“Yes” in S0501) is acquired (homozygous region information acquisition step: S0502). And in case that a continuous probability of polymorphic markers included in the homozygous region information mentioned above satisfies the homologous determination conditions (“Yes” in S0503), a homozygous region is determined as being a homologous region (homologous region determining step: S0504).

The aforementioned process is not restricted to performance via the homologous region determining device of the present invention, and may be undertaken manually. The same applies to the following homologous region determining device.

### Effect of the First Embodiment

In case that human DNA that gives rise to a disease regarding which a causal gene has not yet been identified is used as a sample, it can be said that there is a high possibility that a homologous region determined via the homologous region determining method of the embodiment corresponds to a region with an affected gene. In the same manner as in the case of a human being, in case that DNA of animals or plants is used as a sample, it can be said that there is a high possibility that a region determined as being a homologous region is a region with a disease susceptibility gene. Additionally, via the homologous region determining method, it is possible to easily specify a candidate for a disease susceptibility gene with a smaller number of samples than that necessary with currently existing analysis methods. Furthermore, in case that a region is determined as being a homologous region in relation to sample DNA that does not give rise to a disease, it can be determined that such region is vulnerable in regards to recessive genes.

### Second Embodiment

### Configuration of the Second Embodiment

Explanations are hereinafter given with reference to the second embodiment. An example of a functional diagram of the embodiment is shown in FIG. 6. The homologous region determining device (0600) of the embodiment comprises the polymorphic marker selection section (0601), the homozygosity determining section (0602), the homozygous region information acquisition section (0603), and the homologous region determining section (0604).

The polymorphic marker selection section (0601) is configured so that polymorphic markers as the subject of determination regarding homozygosity are selected from among polymorphic markers of sample DNA indicating a state of diploidy or polyploidy. “Polymorphic markers as the subject of determination regarding homozygosity” refers to the polymorphic markers that execute determination at the homozygosity determining section in regards to a subsequent section among DNA polymorphisms. It is not efficient to determine all polymorphic markers by the homozygosity determining section from the viewpoint of time and cost. Polymorphic markers are not located at equal intervals on chromosomes, and such intervals are varied. Additionally, in regards to use of overly sequential polymorphic markers, there is a high possibility that both such markers are located within the homologous region, which has no importance in relation to identification of the homologous region. Thus, when the polymorphic markers are selected at a certain interval, it can reduce the number of markers to be detected, resulting in a more efficient method. For instance, in regards to selection of polymorphic markers, use of one marker per 5 to 10 kb can be possible. Additionally, it is thought that useful polymorphic markers do not exist in regards to telomeres and centromeres. Thus, such polymorphic markers can be excluded from the subject of determination regarding homozygosity. A database of polymorphic markers has been complied. Therefore, when it is intended to examine all chromosomes for homologous regions, it would be ideal to choose polymorphic markers that are distributed equally over the chromosomes based on the information in the database. Moreover, when a gene region candidate has been specified via associated analysis and affected sib-pair analysis, and the like, polymorphic markers existing within such candidate region are selected in a careful manner. Such selection can further narrow down gene region candidates.

In regards to the homologous determining method of the present invention, in case that the sample DNA is human DNA, if it is intended that SNP be used for polymorphic markers and polymorphic markers are selected from all chromosomes, it is desirable to select 10,000 or more SNPs. Furthermore, to make an even more comprehensive determination, it is desirable to select 100,000 or more SNPs. In such case, a commercially distributed GeneChip (registered trademark) may be used.

One example of a computer-based configuration regarding the polymorphic marker selection section is given as follows. The location information and the homozygosity ratio information regarding polymorphic markers are stored in storage region as a database in advance. Generally speaking, it is said that from thousands of to tens of thousands of polymorphic markers, hundreds of thousands of polymorphic markers, millions of polymorphic markers, or 10,000,000 polymorphic markers exist. Such matters differ according to polymorphic marker type and kind. Therefore, apart from a case in which sufficient resources can be utilized in regards to computer resources, generally, polymorphic markers regarding which homozygosity is determined from the aforementioned polymorphic markers will be selected. In regards to the method of selection, the number of polymorphic markers to be selected is determined in advance, in accordance with given rules, and selection is repeated until the number of the selected polymorphic markers reaches the predetermined number or until given conditions are met based on a value less than or equal to the predetermined number in advance. Such method is adopted. However, selection methods are not limited thereto. Given rules can be the rules by which selection is made so that physical length between polymorphic markers will belong to a given range, or rules by which selection is made so that the homozygosity ratio for a given number of selected and adjacent polymorphic markers will be less than or equal to given values. Also, a rule that one polymorphic marker should be selected per haplotype block via use of haplotype block information may be further added. Furthermore, in case that a region necessary for homologous determination can be selected from all relevant genes based on the purpose of homologous determination, the rules by which selection can be executed within the necessary region are acceptable. At any rate, a selection program, by which the rules for selection from the relevant database are stored in a given storage region and are developed in the main storage region, and by which execution takes place via CPU, selects the aforementioned rules and executes selection of relevant makers from polymorphic marker databases in accordance with such rules. The polymorphic markers selected in accordance with given rules are stored in the storage region for polymorphic markers regarding which location information and homozygosity ratio information have been selected. A large piece of data stored in such storage region is called “the selected polymorphic marker file.” In addition, it is not necessary to execute such selection process every time the homozygosity determining step as below is executed. As long as selection is made in advance, the same selected polymorphic marker file may be used based on type or based on purpose of homologous determination.

The homozygosity determining section (0602) is configured to determine whether the bases making up the polymorphic markers selected by the polymorphic marker selection section (0601) mentioned above indicate homozygosity or not. The determining method is performed in the same manner that of the first embodiment. Processing of other sections is the same as that of the first embodiment. Thus, a description of such processing is omitted here. One example of a computer-based configuration regarding the homozygosity determining section is the same as that of the first embodiment except for the use of a selected polymorphic marker file in lieu of a polymorphic marker file.

The homozygous region information acquisition section (0603) is configured so that from among the polymorphic markers as subjects of determination by the homozygosity determining section (0602) mentioned above, the homozygous region information showing sample DNA in which polymorphic markers which have been determined as corresponding to a state of homozygosity are contiguous is obtained. Explanations are given with reference to FIG. 3B hereinafter. FIG. 3B shows polymorphic markers that exist on DNA (0301). A black bar shows the polymorphic marker for homozygosity, a white bar shows the polymorphic marker for a heterozygote, and a downward pointing triangle above the polymorphic marker shows a selected polymorphic marker. The portions indicated with “0303” in FIG. 3B (i through m) contain polymorphic markers that are not selected (j and l). However, in regards to the present invention, only sequence of the selected polymorphic markers (i, k, and m) is observed. Therefore, the portions “0303” are determined as the regions in which polymorphic markers that have been determined as corresponding to a state of homozygosity are contiguous. That is to say, regardless of whether or not the polymorphic markers that have not been selected from among polymorphic markers that have been determined as corresponding to a state of homozygosity correspond to a heterojunction region or not, it is determined that the aforementioned portions correspond to a homozygous region. Thus, the shaded DNA region FIG. 3B are determined as a homozygous region.

### Description of the Second Embodiment

FIG. 7 shows a description of processes of the homologous region determining method of the second embodiment. First of all, the polymorphic markers as the subject of determination regarding homozygosity are selected from the polymorphic markers of sample DNA indicating a state of diploidy or polyploidy (polymorphic marker selection step: s0701), and determines whether the bases making up the polymorphic markers selected by the polymorphic marker selection step indicate homozygosity or not (homozygosity determining step: S0702). Subsequently, the homozygous region information showing the sample DNA region in which the polymorphic markers that have been determined as corresponding to a state of homozygosity (“Yes” in S0702) by the homozygosity determining step mentioned above are contiguous is acquired (homozygous region information acquisition step: S0703). Furthermore, when the continuous probability of the polymorphic markers included in the homozygous region information mentioned above satisfies the given homologous determination conditions (“Yes” in S0704), a homozygous region is recognized as a homologous region (homologous region determining step: S0705).

### Effect of the Second Embodiment

Based on the homologous region determining method of the embodiment, selection of the polymorphic markers can omit detection of more than a sufficient number of polymorphic markers. Thus, the homologous region can be specified in an efficient manner from the viewpoint of time and costs. Moreover, when a gene region candidate has been specified via associated analysis or affected sib-pair analysis, and the like, selection of the polymorphic markers existing within the gene region candidate in a detailed manner can allow the gene region candidate to be narrowed down further.

### Third Embodiment

### Configuration of the Third Embodiment

A third embodiment of the present invention is explained hereinafter. An example of a functional diagram of the embodiment based on the first embodiment is provided in FIG. 8. The homologous region determining device (0800) of the embodiment comprises a homozygosity determining section (0801), a homozygous region information acquisition section (0802), a homologous region determining section (0803), the homologous region information preservation section (0804), and a homologous region information preservation section (0805).

The homologous region information preservation section (0804) is configured so that multiple pieces of the homologous region information showing a region that has been determined as being a homologous region by the homologous region determining section (0803) are preserved in response to multiple samples. “Homologous region information” refers to information showing a region that has been determined as being a homologous region by the homologous region determining step mentioned above. For example, such information includes the location of a homologous region, continuous probability thereof, continuous distance thereof, location of polymorphic markers included in a homologous region, and ID, and the like. The homologous region information acquisition section preserves the homologous region information for multiple samples.

The homologous region overlapping frequency information acquisition section (0805) is configured so that the homologous region overlapping frequency information showing the overlapping frequency among multiple samples in regards to specific homologous regions is acquired based on homologous region information for multiple samples preserved by the homologous region information preservation section (0804). “Overlapping” means that a homologous region per sample matches a whole or a part of a homologous region regarding another sample. “Overlapping frequency” refers to the number of samples that exhibit overlapping among all samples in regards to homologous regions when multiple samples' homologous regions are overlapped. “Homologous region overlapping frequency information” refers to information showing overlapping frequency among multiple samples in regards to specific homologous regions. For instance, such information includes the location of an overlapping homologous region, overlapping frequency, location of polymorphic markers included in a homologous region, and ID, and the like. Explanations are given with reference to FIG. 9. FIG. 9 shows homologous regions (shaded portions) on the DNA of 4 samples from A through D. The homologous region preservation section preserves the homologous region information of each sample. For instance, the homologous region information in A includes information that regions “1” through “2”, and “3” through “4” are the homologous region. When the homologous region information regarding 4 samples is overlapped, the homologous regions are classified into the regions a through l, and the overlapping frequency for each region is computed. In relation to b, f, i, and k of FIG. 9, only one sample out of four samples is determined as being the homologous region, and thus overlapping frequency is “1.” Computation is made in the same manner. And c, d, and g correspond to 3, h corresponds to 3, and e corresponds to 4. In the case of a sample from each patient to whom the same recessive gene disease has occurred, it can be said that there is the highest possibility that a causal gene for the disease would exist within a region as shown in e in which the overlapping frequency is high.

One example of a computer-based configuration regarding the homologous region information preservation section and the homologous region overlapping frequency information acquisition section is as follows.

As described above, the homologous region file contains location information showing a region in which the computed probability is smaller than that determined under homologous determination conditions as location information showing the homologous region. The homologous region information preservation section records the homologous region files for all samples.

The homologous region overlapping frequency information acquisition section acquires common location information from the homologous region files in regards to multiple samples preserved in the homologous region information preservation section. The common location information is related to frequency of appearance in regards to samples with common location information, and the resulting information is preserved. That is to say, in case that the location information associated with “A to B” (A and B the location of polymorphic markers) is included in a homologous region file for a specific sample, the location information for “A to B” is included in a homologous region file for another separate sample, and homologous region files for 100 samples in total have “A to B” as common location information, the information for “a region of A to B” and the information for “100” are associated with each other and such associated information is preserved. Such an associated and preserved file is called a “homologous region overlapping frequency file.” First, in regards to a computer program, “1” is allocated to the location information showing the polymorphic markers contained in each homologous region file, and such information is preserved. Subsequently, each sample is sequentially searched for. When “1” is allocated to the same location information in regards to the second sample, “1” is added to the location information as a value, and “2” is allocated. When “1” is allocated to the same location information in regards to the third sample, “1” is further added, and “3” is allocated. When the same location information is not included in a homologous region file in relation to the fourth sample, “1” is not allocated. Thus, “0” is added to “3” allocated to the aforementioned information or “3” is kept as it is without executing addition processing. This process is repeated for all samples. The cumulative value to which such value of “1” is added for all samples is obtained. In relation to the location information that is not contained in a homologous region file for each sample, “0” may be allocated as a value related to the location information for such sample, and such “0” value may be added. Alternatively, it is acceptable for addition processing not to be executed.

The cumulative value is associated with the location information and is recorded in a homologous region overlapping frequency file. Also, in case that a homologous file is added, “1” is allocated to the location information concerning polymorphic markers included in the added homologous region file, and such information is preserved. And due to adding such information to the recorded homologous region overlapping frequency file, a new homologous region overlapping frequency file is generated. At this time, the previous homologous region overlapping frequency file is deleted. With the outputting of a final homologous region overlapping frequency file, it is possible to determine overlapping frequency of a homologous region.

Additionally, in case that there are errors in regards to an overlapped homologous region file or in the case of reduction of the number of files, the processing resulting when “1” allocated to the location information showing the polymorphic markers in the homologous region files that are intended to be extracted from the homologous region overlapping frequency files is subtracted is executed.

### Description of the Third Embodiment

FIG. 10 shows a description of processing of the third embodiment. First of all, it is determined whether the bases making up polymorphic markers of sample DNA indicating a state of diploidy or polyploidy indicate homozygosity or not (homozygosity determining step: S1001). Subsequently, from among the polymorphic markers that have become the subject of determination via the homozygosity determining step mentioned above, the homozygous region information showing the sample DNA region in which polymorphic markers which have been determined as corresponding to a state of homozygosity are contiguous is acquired (homozygous region information acquisition step: S1002). And when the continuous probability of the polymorphic marker included in the homozygous region information mentioned above satisfies the homologous determination conditions (“Yes” in S1003), the homozygous region is determined as being a homologous region (homologous region determining step: S1004). Furthermore, the homologous region information showing the region that has been determined as being a homologous region by the homologous region determining step is acquired for multiple samples (homologous region information acquisition step: S1005). Based on the homologous region information for multiple samples that has been acquired by the homologous region information acquisition step mentioned above, the frequency of occurrence of overlapping specific homologous regions among multiple samples is obtained (homologous region overlapping frequency acquisition step: S1006).

### Effect of the Third Embodiment

According to the homologous region determining method of the embodiment, when human DNA, regarding which identification of a causal gene has not been conducted, is used as a sample, it is possible to narrow down a region that has a high possibility of having a disease causal gene. The same applies to the search for disease susceptibility genes for animals and plants. Additionally, upon performance of breed improvement operations for plants and animals such as livestock and the like, with the homologous region determining method of the embodiment, it is possible to search for genes regarding which recessive and changeable functions or characteristics are likely to occur.

### Fourth Embodiment

### Configuration of the Fourth Embodiment

Explanations are given in regards to the fourth embodiment hereinafter. An example of a functional diagram of the embodiment based on the first embodiment is shown in FIG. 11. The homologous region determining device (1100) of the embodiment comprises a homozygosity determining section (1101), a homozygous region information acquisition section (1102), a homologous region determining section (1103), a homologous region information preservation section (1104), a homologous region overlapping frequency information acquisition section (1105), a homologous region information accumulation section (1106), and an important homologous region information acquisition section (1107).

The homologous region information accumulation section (1106) is configured such that the overlapping frequency obtained through the homologous region overlapping frequency acquisition section (1105) mentioned above is adjusted to the homologous region information, and such that the resulting information is accumulated. “Adjusted to” refers to “together with.” That is to say, the homologous region information accumulation section accumulates the location of a homologous region, continuous probability regarding the existence thereof, continuous distance thereof, location of polymorphic markers included in a homologous region, ID, and the like in conjunction with the aforementioned information.

The important homologous region information acquisition section (1107) is configured so that from among the homologous region information accumulated in the homologous region information accumulation section (1106) mentioned above, the homologous region information associated with a frequency that is greater than or equal to a given overlapping frequency is acquired. “Given overlapping frequency” refers to an established overlapping frequency. For example, such given overlapping frequency is established as “10.” “Homologous region information” refers to homologous region information to which more than or equal to given overlapping frequency is adjusted. In case that homologous regions for 30 samples are determined, if the given overlapping frequency is “10,” from among the homologous region information accumulated in the homologous region information accumulation section, only the homologous region information determined as being the homologous region for 10 or more samples out of 30 samples can be obtained.

One example of a computer-based configuration regarding the homologous region information accumulation section and the important homologous region information acquisition section is as follows.

The homologous region information accumulation section preserves a homologous region overlapping frequency file with which location information obtained by the homologous region overlapping frequency acquisition section mentioned above is associated in the storage region. Additionally, the homologous region overlapping frequency file may be stored with information relating to birthplace, habitat, disease, race, variety, or the like, and may be stored as a separate file classified by the aforementioned items.

From among the homologous region overlapping frequency files with which the location information stored in the homologous region information accumulation section mentioned above is associated, the important homologous region information acquisition section acquires information the homologous region information to which more than or equal to given overlapping frequency is adjusted. Such the homologous region information to which more than or equal to given overlapping frequency is adjusted is called a “homologous region file.” That is to say, in relation to the homologous region overlapping frequency file, in case that the information “A:20, B:50, and C:100 . . . (where all excluded values are 100), Y:50, Z:30” (where A:20 represents the fact that polymorphic markers with A's location are included in the “20's” homologous region overlapping file) is stored, if homologous region information in which overlapping frequency is greater than or equal to 50 is specified, the location information of “from B to Y” is recorded in an important homologous region file. Ultimately, when the location information stored in the important homologous region file is outputted, it is possible to specify the important homologous region.

Also, genetic information is associated with location information, and such information is separately stored in the storage region in the form of a genetic information file. “Genetic information” refers to information regarding a protein encoded by genes. If a relationship with a disease is known, genetic information is associated with information pertaining to disease names, and the like. In regards to such genetic information file, the existing database and output data are obtained via communications and recording media, and may be stored in a storage region, such as a hard disk drive or RAM. In case that location information regarding the homologous region overlapping frequency file includes a region in which recessive genes separately stored in the storage region exist, such genetic information may be associated with the homologous region overlapping frequency file and may be stored.

### Description of the Fourth Embodiment

FIG. 12 shows a description of processing of the fourth embodiment. First of all, it is determined whether the bases making up polymorphic markers of sample DNA indicating a state of diploidy or polyploidy indicate homozygosity (homozygosity determining step: S1201). Subsequently, from among polymorphic markers that have been subjects of determination by the homozygosity determining step mentioned above, homozygous region information showing a region of sample DNA in which polymorphic markers which have been determined as corresponding to a state of homozygosity are in sequence is acquired (homozygous region information acquisition step: S1202). And in case that the continuous probability of polymorphic markers included in the homozygous region information mentioned above satisfies the homologous determination conditions (“Yes” in S1202), the homozygous region is determined as being a homologous region (homologous region determining step: S1204). Furthermore, homologous region information showing a region that has been determined as being a homologous region by the homologous region determining step is acquired for multiple samples (homologous region information acquisition step: S1205). Based on the homologous region information for multiple samples that have been acquired by the homologous region information acquisition step mentioned above, the frequency of specific homologous regions overlapping among multiple samples is obtained (homologous region overlapping frequency acquisition step: S1206). Finally, the overlapping frequency obtained through the homologous region overlapping frequency acquisition section mentioned above is adjusted to the homologous region information, and the resulting information is accumulated (the homologous region information accumulation step: S1207). From among the homologous region information accumulated by the homologous region information accumulation step mentioned above, the homologous region information to which more than or equal to given overlapping frequency is adjusted is obtained (important homologous region information acquisition step: S1208).

### Effect of the Fourth Embodiment

Via the homologous region determining method of the embodiment, from among the regions determined to be homologous regions in multiple samples, only the regions in which overlapping frequency is far higher can be obtained. When regions involving searching for disease susceptibility genes are narrowed down, due to changes in a set values for given overlapping frequency, adjustment of the number of candidate regions to be searched for can be possible.

### Fifth embodiment

### Configuration of the Fifth Embodiment

Explanations are given with reference to the fifth embodiment. An example of a functional diagram of the embodiment based on the first embodiment is shown in FIG. 13. The homologous region determining device (1300) of the embodiment comprises a homozygosity determining section (1301), a homozygous region information acquisition section (1302), a homologous region determining section (1303), and a homologous region information output section (1304).

The homologous region information output section (1304) is configured so that the homologous region information as information showing the homozygous region determined to satisfy the homologous determination conditions by the homologous region determining section (1303) is visualized and outputted. “Visualized and outputted” refers to tangible representation. For instance, relevant information can be outputted in the form of tables, graphs, or figures. Outputting can be undertaken by making indications on a display, by print-out, via writing using recording media, and the like. Outputting of visual homologous region information allows for easy determination of the location of a homologous region in a sample.

One example of a computer-based configuration regarding the homologous region information output section is as follows. A homologous region file obtained by the homologous region determining section is outputted from the homologous region output section via the input and output interface. The location information regarding homologous regions stored in the homologous region file is read out sequentially, and the process of visualization of regions on the chromosomes corresponding to the location information is undertaken in accordance with the relevant rules. Such rules may be rules stipulating that the location information for both ends of the homologous region is arrayed starting with the location information corresponding to the lowest number based on numeric order of chromosomes, or may be rules stipulating that 100 kb of the length of a homologous region corresponds to a region with 1-mm width and that the resulting region be illustrated on a chromosome map. As examples, FIGS. 17A, 17B, and 17C show what has been outputted on a chromosome map. Black regions indicate homologous regions. FIGS. 17A, 17B, and 17C indicate homologous regions for three individuals. Chromosome locations as homologous regions differ from each other. And it is understood that through visualization, homologous regions serve a function in relation to individual fingerprint.

### Description of the Fifth Embodiment

One example of a description of processing of the fifth embodiment through a computer-based configuration is explained with reference to FIG. 20. In FIG. 20, SNP is used as a polymorphic marker. And as a homologous determination condition, the continuous probability is set as being less than or equal to 1/105. First of all, when an SNP typing result is obtained, SNP types are divided into four categories of AA homo, BB homo, AB hetero, and Nocall, and 1, 2, 3, and 4 apply thereto respectively (S2001). The base that is indicated in regards to A and B must be determined in advance. “Nocall” means that the relevant base could not be detected. SNP is changed to be aligned based on relevant chromosomes and locations (S2002). And one piece of the information corresponding to the lowest value in a numeric order of chromosomes that has not been processed is selected (S2003). Types of polymorphisms are searched for from among the selected chromosome corresponding to the lowest value in a numeric value (S2004). First of all, SNP of 1 or 2 as the “start” of a homozygous region is searched for (S2005-S2007). The SNP corresponding to homozygosity that is detected first is deemed to be the “start.” (S2008). Subsequently, an adjacent SNP is searched for (S2009). And if the SNP corresponds to “4,” the subsequent SNP is searched for (S2010). In case that the adjacent SNP is “1” or “2” (“Yes” in S2011), the homozygosity concerning SNP regarding sequential homozygosity is multiplied (S2012). Also, if the adjacent SNP is “3” (S2013), one SNP before the SNP is deemed to be “end” of the homozygous region (S2014). In case that all processes concerning the selected chromosomes are not finished (“No” in S2015), a step to search for SNP as being the “start” of homozygous regions (S2006), such action is repeated until all SNPs concerning the selected chromosomes are searched for. Subsequently, the process returns to the step of searching for SNP as the “start” of a homozygous region (S2015), and the relevant process is repeated until all SNPs for the selected chromosomes have been searched for. All SNPs for the selected chromosomes are searched for (“Yes” in S2015), and it is confirmed whether or not processing of all chromosomes has been completed. In case that such processing is not finished yet (“No” in S2016), the searching of the next chromosome commences (S2003). When the processing of all chromosomes is finished (“Yes” in S2016), only the information concerning a region in which the value by which the homozygosity ratio is multiplied satisfies the homologous determination conditions (less than or equal to 1/105) is recorded and outputted (S2017).

### Effect of the Fifth Embodiment

Visualization of homologous region information can easily allow comparison with the location of an affected gene and comparison with other samples. Additionally, in case that a long homologous region exists, it is easy to discover the fact that consanguineous marriage took place within close family lines. In case that there exist only short homologous regions, it is easy to determine that no consanguineous marriage has taken place within close family lines.

### Sixth Embodiment

### Configuration of the Sixth Embodiment

Explanations are given with reference to the sixth embodiment. An example of a functional diagram of the embodiment based on the second embodiment is shown in FIG. 14. The homologous region determining device (1400) of the embodiment comprises the polymorphic marker selection section (1401), a homozygosity determining section (1402), a homozygous region information acquisition section (1403), a homologous region determining section (1404), a homologous region information preservation section (1405), a homologous region overlapping frequency information acquisition section (1406), a homologous region information accumulation section (1407), a important homologous region information acquisition section (1408), a homologous region overlapping frequency visualization information output section (1409), and a homologous region information output section (1410).

The homologous region overlapping frequency visualization information output section (1409) is configured so that the homologous region overlapping frequency visualization information corresponds to visualized and outputted homologous region overlapping frequency information obtained by the homologous region overlapping frequency information acquisition section. Outputting of visualized homologous region overlapping frequency information can allow easy determination as to the location of a homologous region with high overlapping frequency.

One example of a computer-based configuration regarding the homologous region overlapping frequency visualization information output section is as follows. An overlapping frequency file obtained by the homologous region information overlapping frequency acquisition section is outputted by the homologous region overlapping frequency visualization information output section via the input and output interface. The location information regarding homologous regions stored in the overlapping frequency file is read out sequentially, and the process of visualization concerning regions on the chromosomes corresponding to the location information is undertaken in accordance with the relevant rules. Such rules may be rules in which outputting takes place based on a graph under a condition such that a horizontal axis indicates the chromosome location and the vertical axis indicates overlapping frequency. As an example of a method of outputting, FIG. 19 shows the output on a chromosome map that involves relating the overlapping frequency to color density. Darker regions indicate homologous regions with high overlapping frequencies. It is easy to determine that a region indicated by an arrow corresponds to a region with a high overlapping frequency.

The homologous region information output section (1410) is configured so that so that the homologous region information to which more than or equal to given overlapping frequency is adjusted and such information is obtained by the important homologous region information acquisition section is visualized and outputted. Outputting of important visualized homologous region information can allow for easy determination as to where homologous region of more than the established high overlapping frequency is.

One example of a computer-based configuration regarding the important homologous region information output section is as follows. An important homologous region file obtained by the important homologous region information acquisition section is outputted by the important homologous region information output section via the input and output interface. The location information regarding homologous regions stored in the important homologous region file is read out sequentially, and processing of visualization concerning regions on the chromosomes corresponding to the location information is undertaken in accordance with the relevant rules. Such rules may be the rules by which the location information concerning the homologous region is arrayed from the information corresponding to the lowest value in a numeric order of chromosomes, or may be rules by which 100 kb of the length of important homologous region correspond to a region with 1-mm width, and the resulted region is illustrated on a chromosome map. As an example of the methods of outputting, from among 2 samples of FIGS. 17A and 17B, the outputted important homologous region information under a condition in which the overlapping frequency corresponds to 2 is shown in FIG. 18D. From among the three samples of FIGS. 17A, 17B, and 17C, the information in which the important homologous region information was inputted under the condition that the overlapping frequency corresponds to “3” is shown in (E) of FIG. 18.

### Effect of the Sixth Embodiment

The homologous region information is outputted as homologous region overlapping frequency visualization information or important homologous region information. Due to such outputting, it is possible to clarify the frequency of occurrence of a homologous region for a relevant group. The homologous region determining device with the homologous region overlapping frequency visualization information output section can allow easy determination concerning regions with the high overlapping frequency. The homologous region determining device with the important homologous region information output section can output only information corresponding to a homologous region with an established overlapping frequency or more. Thus, it is possible to restrict the region related to a gene search and to undertake efficient gene screening.

### Seventh Embodiment

Explanations are given with reference to the seventh embodiment. The embodiment corresponds to a gene screening method with specific functions in which genetic sequences included in the homologous regions determined by the homologous region determining methods or homologous region determining devices mentioned in one of the above descriptions are identified and are compared with sequences of normal genes.

This gene screening method is used to determine gene sequences within a region determined as being a homologous region and to compare the same with the sequences of normal genes. Thereby, gene sequences abnormalities in sample DNA are examined. In case that a sample DNA corresponding to a recessive gene disease for which the casual gene has not been known at all is used, regions determined as being homologous regions are candidate regions for the locations of disease susceptibility genes. Conducting detection of all gene sequences within a candidate region allows specification of disease susceptibility genes. That is to say, in case that abnormal genes exist in sample DNA corresponding to the same disease, such genes can be specified as causal genes. Moreover, even under strict homologous determination conditions, when identification of gene sequences in a region determined as being a homologous region is conducted, it is possible to efficiently specify disease susceptibility genes.

Additionally, a homologous region is a region between polymorphic markers that have been determined as corresponding to a state of homozygosity. Thus, it is possible for the genes extending from the polymorphic markers indicating homozygosity of both ends thereof to the polymorphic markers determined as being the heterojunction subsequently to be homologous as a matter of fact. Therefore, when gene screening is undertaken, it is desirable to detect genes extending up to the polymorphic markers determined as being the heterojunction subsequently.

### Eighth Embodiment

Explanations are given with reference to the eighth embodiment. The embodiment corresponds to a gene screening method with specific functions. According to this method, the homologous regions identified by the homologous region determining methods or homologous region determining devices mentioned in one of the above descriptions are overlapped with the homologous region for which information is accumulated in the homologous region information accumulation section, and the gene sequences included in the overlapping region are identified and compared with the sequences of normal genes.

In case that the homologous region information regarding sample DNA that may or may not correspond to a disease is overlapped with the homologous region information that is connected with the disease information accumulated in the homologous region information accumulation section, gene sequences included in the overlapping region are identified and compared with the sequences of normal genes. Thereby, it can be determined whether a disease exists or not. The homologous region information accumulation section relates the location information concerning genes that could cause disease or genes that could cause significant characteristics to the homologous region information, and accumulates the resulted information. Due to this, it is possible to use the same for genetic diagnosis.

### Ninth Embodiment

Explanations are given with reference to the ninth embodiment. The embodiment corresponds to a gene screening method with specific functions. And according to this method, it is determined whether or not the homologous regions determined by the homologous region determining methods or homologous region determining devices mentioned in one of the above descriptions could contain genes that have already been known to function in a homozygous state. In the case of a region that could contain a gene that has been already known, sequences of corresponding known genes and corresponding genes of sample DNA are compared.

“Functions” may correspond to dominant characteristics as well as recessive characteristics. For instance, characteristics of being resistant to the cold or pests or characteristics of having a high sugar content are possible with homozygosity. In case that a homologous region of sample DNA is overlapped with a gene that is already known to serve its function by being homozygous, the sequence of genes included in the overlapping region is identified and compared with the sequences of normal genes. Thereby, it is possible to examine the existence of corresponding genes. For instance, comparing a corresponding region with a casual gene region of a recessive gene, a simple recessive gene disease can be diagnosed. In case that a sample's homologous region is overlapped with an affected gene region, the sequences of genes are identified and casual genes are specified.

### Tenth Embodiment

Explanations are given with reference to the tenth embodiment. The embodiment corresponds to a gene screening method with specific functions. And according to this method, in case that the aforementioned sample DNA corresponds to a disease, in case that the homologous regions determined by the homologous region determining methods or homologous region determining devices mentioned in one of the above descriptions contain a gene that is expected to be related to a corresponding disease, the sequences of the corresponding genes in the homologous region of the sample DNA mentioned above are identified and compared with normal genes.

With the gene screening method of the following example, a causal gene for alveolar microlithiasis mentioned in the embodiment as below is identified. Details concerning this screening method are stated in the example.

### Example 1

Detailed explanations are given by using examples of the identification of the causal gene for alveolar microlithiasis. However, the present invention is not limited to such examples.

<Alveolar Microlithiasis>

Alveolar microlithiasis is a disease in which an unlimited number of fine stones composed of laminated and growth-ring-shaped layers of calcium phosphate are formed within the alveoli. It is a rare disease with unknown causes (non-patent document 6). This disease can be discovered from childhood to adulthood. However, there is no gender difference in regards to the onset of the disease. The symptoms differ by age. Normally, according to the cases discovered in the period from childhood through early adulthood, remarkably diffuse lung shadows can be discovered via chest x-ray. Despite the fact, generally, patients are not aware of the symptoms. However, patents who are over 40 years old notice symptoms such as breathing difficulties or coughing during exercise. The long-term prognosis concerning this disease differs based on age at the time of discovery thereof. However, the prognosis is not always good. In particular, for middle-aged patients who are over 40 years old, as symptoms progress, respiratory symptoms such as coughing, breathing difficulties, or the like take place. Furthermore, some patients die of respiratory failure as the symptoms progress.

The frequency of occurrence of this disease among siblings is high, and a tendency of horizontal transfer, such as among brothers and sisters, can be discovered. Thus, it is thought that such disease is a genetic lung disease based on autosomal recessive inheritance (non patent document 7). However, the relevant causal gene has not yet been identified. This is a rarely occurred disease. However, it can be said that potential frequency concerning the onset of such disease is high in the countries in which numbers of siblings are high, such as an insular country with a racially homogeneous population, or in counties in which the percentage of marriages accounted for by consanguineous marriages is high as a result of religious background. Thus, this disease cannot be ignored. In particular, in Japan, it is known that the number of cases of this disease are high compared with the rest of the world (non-patent document 8). Thus, investigation into the cause thereof and into treatment methods therefor is desired. However, effective methods of treatment other than relevant treatment such as oxygen therapy and lung transplantation have remained unknown.

<Sample>

DNA samples from 5 patients who started alveolar microlithiasis shown in FIG. 15 were used. Diagonal lines show the dead patients. Patients 1, 2, and 4 correspond to a family with consanguineous marriage, and there are patients with alveolar microlithiasis within the family line. Also, patent 3 does not belong to a family line exhibiting consanguineous marriages, but there is a patient with alveolar microlithiasis within such patient's family line. Patient 5 is not known as to whether this patient is from a consanguineous marriage line. In regards to a sample DNA, adjustment was made based on blood for living patients. And adjustment was made based on paraffin-embedded tissues samples for dead patients. As a method for extracting genome DNA, any publicly known method can be used.

Using examples from a case of phenol treatment from blood, explanations are provided hereinafter. Lysis buffer (final concentration: 100 μg/ml, Proteinase K, 50 mM Tris-HCL (pH 7.5), 10 mM CaC 12, 1% SDS) was added to 5 ml of corresponding peripheral blood. The resultant was incubated for 30 minutes at 50□, and cells were dissolved. Subsequently, phenol that had been saturated with TE buffer was added to the aforementioned cell lysate. Thereafter, a container was rotated several times, and the content was mixed. Subsequently, centrifugal treatment was conducted for 10 minutes at 3,000×g at room temperature. And the contents were separated into a water layer and phenol layer. Only the top water layer was extracted, and it was transferred to a new container. Again, an equal amount of phenol-chloroform mixture (mixing ratio 1:1) was added to such water layer. The container was rotated several times, and mixing was conducted. Next, centrifugal treatment was conducted for 10 minutes at 3,000×g at room temperature again. The contents were separated into the following three layers: water layer, interlayer (denatured protein layer), and phenol-chloroform layer. Then, only the water layer was extracted so that denatured proteins making up the interlayer would not be mixed therewith. Thereafter, until it became impossible to identify the interlayer, the aforementioned phenol-chloroform mixture treatment was repeated several times. Next, PNase A was added to the water layer sample obtained at the last stage so that the final concentration corresponded to 50 μg/ml. The resultant was incubated for 1 hour at 500, and RNA was dissolved. Subsequently, the aforementioned lysis buffer was added, Proteinase K treatment was undertaken, and RNase A in the water layer was deactivated. And an equal amount of the aforementioned phenol-chloroform mixture was added, and phenol-chloroform treatment was conducted. 1/10 of the content of sodium acetate and an equal amount of isopropanol were added to the water layer contents after the treatment, and the resultant was gently stirred. Finally, the intended genome DNA was obtained by looping precipitated genome DNA with a glass. Alternatively, the relevant DNA was obtained under after centrifugal treatment was conducted for 10 minutes at 3,000×g at room temperature again.

<Selection of Polymorphic Markers>

Selection of polymorphic markers was conducted using the Affimetrix's GeneChip® Human Mapping 100 k set, which allows evenly distributed allocation over the all chromosomes. The GeneChip Human Mapping 100 k set can broadly cover regions except for telomere and centromere, and can detect about 100,000 SNPs simultaneously. Regions which contain at least one SNP within 100 kb account for 92% of all DNAs, 83% of those within 50 kb, and 40% of those within 10 kb. Thus, this method is desirable for identification of homologous regions when the cause of a disease has not been discovered. In FIG. 16, the SNP coverage region is shown.

<SNP Typing>

SNP typing was conducted in regards to sample DNAs mentioned above. Also, in order to preserve reliability concerning identification, analyses were conducted by the following two companies: the Australian Genome Research Facility and AROS applied biotechnology. The results of typing were remarkably well matched. SNP typing was conducted in accordance with the Affimetrix's GeneChip Mapping 100k Assay Manual.

<Identification of Homozygous Regions>

Based on the results of SNP typing, it was determined whether a relevant region corresponded to a state of homozygosity, and a region in which a sequence indicates homozygosity was identified.

<Identification of Homologous Regions>

Detection of 100,000 SNPs was conducted. Thus, based on the homologous determination conditions that 1/105 as a continuous probability applies, homologous regions were identified. Identification of homozygous regions and homologous regions was conducted through a computer executing programs described with reference to FIG. 23 through FIG. 29 as below. In these figures, homozygous regions were indicated as SHS (Strand the likeh of Homozygous SNPs).

Homologous regions identified as such can be visualized in the form shown in FIG. 17 by the homologous region output section, and related information can be outputted. FIGS. 17A, 17B, and 17 C indicate homologous regions of patients 1, 2, and 3. The parents of patients 1 and 2 of FIGS. 17A and 17B underwent a cross-cousin marriage. Thus, there existed long homologous regions. On the other hand, patient 3 of FIG. 17C, who was not from a family line exhibiting consanguineous marriages, did not have long homologous regions but rather had short homologous regions which seemed to derive from distant ancestors.

<Identification of Important Homologous Regions>

The commonly shared portions of patients 1 and 2, that is to say, the area where the overlapping frequency corresponds to 2 in regards to important homologous regions, is shown in FIG. 18D. Both patients have long homologous regions. Thus, narrowing down candidate regions cannot be conducted. However, the commonly shared portions of 3 samples of patients 1 through 3, that is to say, the area where the overlapping frequency corresponds to 3 in regards to important homologous regions, is visualized and outputted, and it corresponds to FIG. 18D. In this figure, important homologous regions could be narrowed down by patient 3, who was not from a family line exhibiting consanguineous marriages. The total combined length of such important homologous regions was 11.5 Mb. FIGS. 18D and 18E show that important homologous regions were identified by the program mentioned in FIGS. 30 through 33, and the same were visualized and outputted by the homologous region output section.

<Identification of Causal Genes>

Important homologous regions of 11.5 Mb contained 35 genes. Among 35 genes, some of 25 genes were known, or their functions were almost completely known. Of all such genes, only one gene that that coded for a phosphate symporter was a gene that appeared to be directly connected to the pathologic condition of alveolar microlithiasis. Therefore, SLC34A2 was identified as a candidate gene. And exon sequences of SLC34A2 from 5 samples has been examined. The results showed that all genes had homozygous variations. On the other hand, variations were not discovered in the genes of 10 healthy individuals. In regards to base sequences of SLC34A2, considering relevant sequences as primers, by the using BigDye Terminator vl. 1 cycle sequencing Kit (ABI), reactions were allowed to progress in accordance with the protocols attached thereto. It was confirmed that base sequences of the product of a reaction were directly read and that amplified products were altered by the Automatic DNA sequencer (ABI PRISM 310). Additionally, extraction of genome DNA from a healthy individual is conducted with the same method of genome DNA extraction used for the patients above.

5 individuals' SLC34A2 gene alterations were based on the 2 types described below. Furthermore, concerning the altered proteins based on gene alterations in regards to such 2 types, it was revealed that neither thereof had activity as an IIb sodium-phosphate symporter. Based on the results mentioned above, it became clear that the human SLC34A2 gene corresponded to a casual gene for alveolar microlithiasis, and deactivation of functions of the IIb sodium-phosphate symporter was related to an onset of alveolar microlithiasis (non patent document 9).

The first alteration was caused by substitution shown in FIG. 21A. Specifically, in relation to a wild type base sequence (2101), a sequence of 15 bases from T at position number 13290 in SEQ ID: No. 1 to G at position number 13304 was substituted by a sequence of 19 bases indicated as SEQ ID: No. 2 in regards to an altered-type base sequence (2192). Frameshift is caused by such alteration, and thus, a stop codon emerges in the midst of a wild type DCS. As a result, a stop codon emerges, and an amino acid altered type human iib sodium-phosphate symporter protein (2105) making up 313 amino acids emerges. 5 transmembrane domains (TM) on the end side of C from among 8 TMs that are expected based on the sequence of amino acids of a wild type protein (2104) are lost from such altered type protein.

The second alteration was also caused by the substitution shown in FIG. 22 A. Specifically, in regards to a wild type base sequence (2201), GT, which was indicated as a splicing donor site (double underline portion) caused a mutation such that such GT was substituted by AT in regards to a altered-type (2202). Based on such alteration, after such gene transcription, the 8^{th} intron could not be removed by mRNA splicing. Due thereto, as shown in FIG. 22B, mature mRNA corresponds to a base sequence where the alteration in the 8^{th} intron remained. That is to say, a wild type CDS (cording sequence: amino acid sequence) corresponds to the same condition as that of the base sequence indicated as from T at position number 14303 in SEQ ID: No. 4 to G at position number 15670. Frameshift is caused by such alteration, and thus, a stop codon emerges within the base sequences. As shown in FIG. 22, amino acid altered type human iib sodium-phosphate symporter protein (2204) making up 359 amino acids emerges. As shown in FIG. 22C, 5 transmembrane domains (TM) on the end side of C from among 8 TMs that are expected based on the sequence of amino acids in regards to a wild type protein (2104) are lost in regards to such altered type protein.

Here, the sequence number 1 indicates all base sequences on the genome of wild type human SLC34A2 gene (5′ untranslated region and 3′ untranslated region) and the amino acid sequence corresponding to the coding regions thereof. The location information concerning the numbers of exons and introns mentioned above is described within the sequence listing. The SEQ ID: No. 2 indicates CDS regarding the SEQ ID: No. 1 mentioned above. The SEQ ID: No. 3 indicates the sequence making up 19 bases which are substituted due to the alteration A mentioned above. CDS regarding the sequence number 1 mentioned above. The SEQ ID: No. 4 represents the base sequences of the 8^{th} intron with mutation in a splicing donor site in regards to the aforementioned alteration B.

<Observation>

Based on the results mentioned above, the homozygosity mapping method used for identification of a recessive disease gene using a family line exhibiting consanguineous marriages has been proved to be extensively applicable for patients with a family line that does not exhibit consanguineous marriages. In regards to identification of low-permeability casual genes for alveolar microlithiasis, only 3 samples led to identification of genes. Thus, this fact suggests that it is possible to use the homozygosity mapping method for identification of other affected gene recessive disease genes with a small number of samples. Thus, it has been revealed that the homologous region determining method, homologous region determining device, and gene screening method of the present invention offer a remarkably effective analysis method in regards to identification of recessive genes.

### INDUSTRIAL APPLICABILITY

In regards to research regarding searches for disease susceptibility genes caused by recessive genes that require many family lines and control groups, the homologous region determining method, homologous region determining device, and gene screening method of the present invention allow identification of disease susceptibility genes with a small number of samples (3 samples for alveolar microlithiasis). The present invention makes it possible to identify casual genes with a small number of samples and without the need for family line analysis. Thus, the present invention can be also applied to low-permeability recessive gene diseases in which casual genes have not been identified because of a lack of cases at present. The identified genes will have a high degree of usability in the area of drug discovery. Moreover, due to observation of overlapping frequency in multiple samples, when multiple overlapping regions exist, it is possible to specify multiple candidate regions in regards to disease susceptibility genes. Thus, the present invention can be applied to polygenic diseases. In regards to a sample without diseases and a family line exhibiting consanguineous marriages, with identification as to whether regions existing recessive genes correspond to homologous regions or not, it is possible to use the present invention for simple diagnoses of recessive gene diseases.

Furthermore, the present invention can be used for identification of recessive genes that serve useful functions and recessive genes that would result in useful characteristics. Thus, there is a high degree of industrial applicability relating to livestock and agriculture.

### BRIEF DESCRIPTION OF DRAWINGS

FIG. 1 is an explanatory diagram relating to the concept of a homologous region.

FIG. 2 is an example of a functional diagram of the first embodiment.

FIG. 3 is an explanatory diagram relating to the concept of a homozygous region.

FIG. 4 is an explanatory diagram relating to the relationship between a homologous region and a polymorphic marker.

FIG. 5 is an explanatory diagram relating to a flowchart of the first embodiment.

FIG. 6 is an example of a functional diagram of the second embodiment.

FIG. 7 is an explanatory diagram relating to a flowchart of the first embodiment.

FIG. 8 is an example of a functional diagram of the third embodiment.

FIG. 9 is an explanatory diagram relating to the concept of homologous region overlapping frequency.

FIG. 10 is an explanatory diagram relating to a flowchart of the third embodiment.

FIG. 11 is an example of a functional diagram of the fourth embodiment.

FIG. 12 is an explanatory diagram relating to a flowchart of the fourth embodiment.

FIG. 13 is an example of a functional diagram of the fifth embodiment.

FIG. 14 is an example of a functional diagram of the sixth embodiment.

FIG. 15 is a family tree of the patients used for the first embodiment.

FIG. 16 is a diagram representing the scope of SNPs selected in connection with the first embodiment.

FIG. 17 is a diagram showing homologous regions of alveolar microlithiasis patients.

FIG. 18 is a diagram showing important homologous regions of alveolar microlithiasis patients.

FIG. 19 is a diagram showing an example of output method for homologous region overlapping frequency.

FIG. 20 is an explanatory diagram relating to a flowchart of the fifth embodiment.

FIG. 21 is a diagram showing causal genes of alveolar microlithiasis involving a first alteration.

FIG. 22 is a diagram showing causal genes of alveolar microlithiasis involving a second alteration.

FIG. 23 shows a program (1) used to identify homozygous regions and homologous regions.

FIG. 24 shows a program (2) used to identify homozygous regions and homologous regions.

FIG. 25 shows a program (3) used to identify homozygous regions and homologous regions.

FIG. 26 is shows a program (4) used to identify homozygous regions and homologous regions.

FIG. 27 shows a program (5) used to identify homozygous regions and homologous regions.

FIG. 28 shows a program (6) used to identify homozygous regions and homologous regions.

FIG. 29 shows a program (7) used to identify homozygous regions and homologous regions.

FIG. 30 shows a program (1) used to identify important homologous regions.

FIG. 31 shows a program (2) used to identify important homologous regions.

FIG. 32 shows a program (3) used to identify important homologous regions.

FIG. 33 shows a program (4) used to identify important homologous regions.

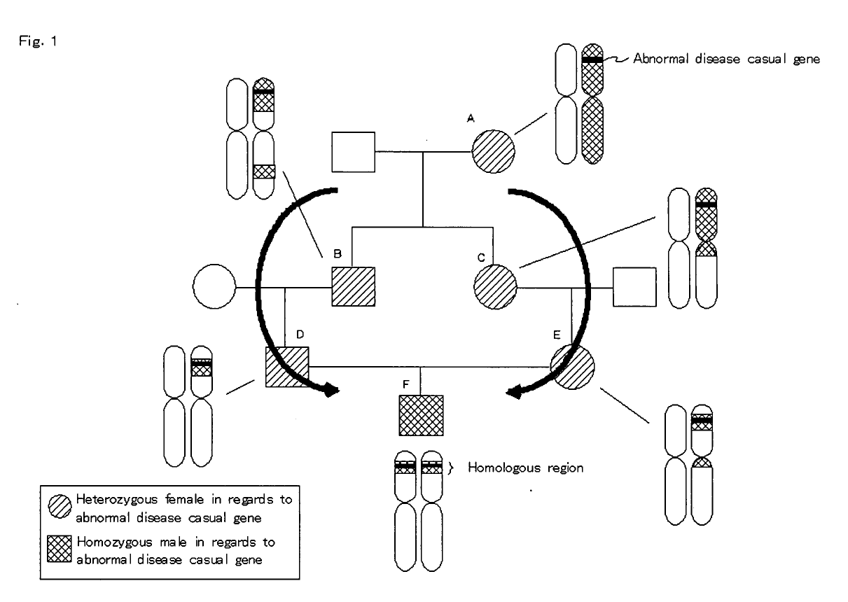

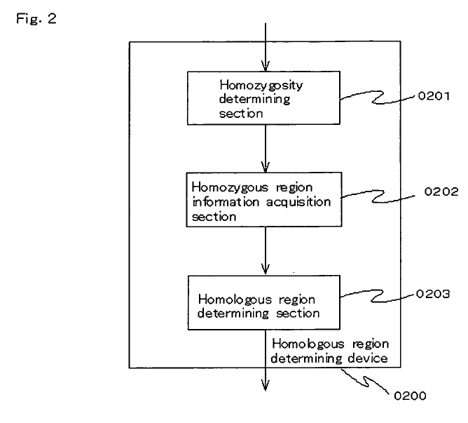

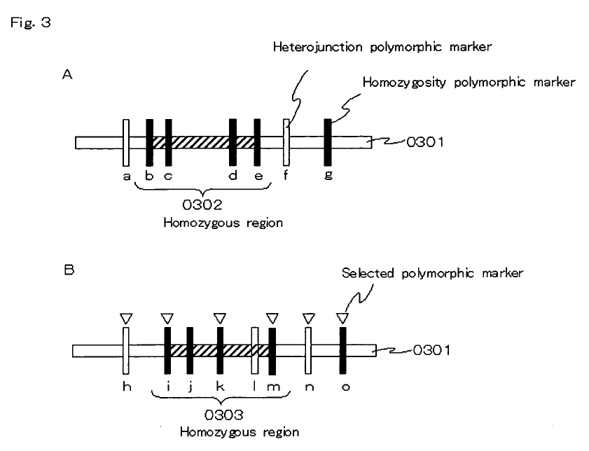

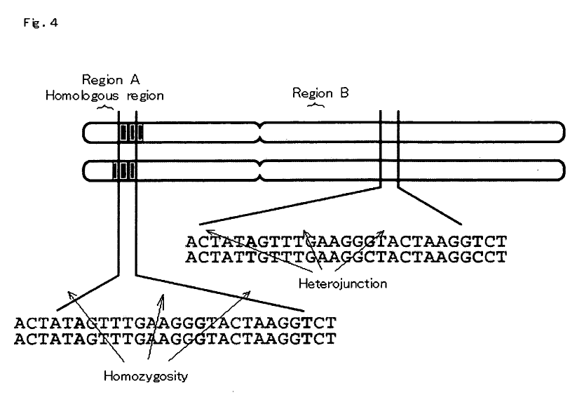

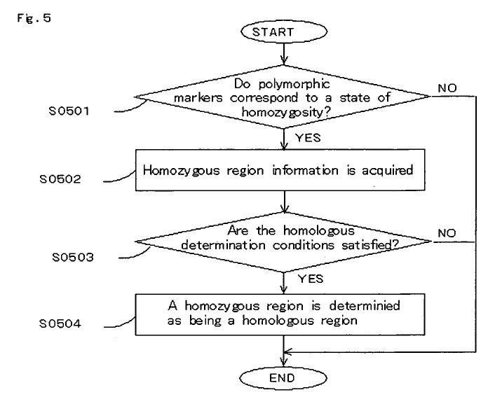

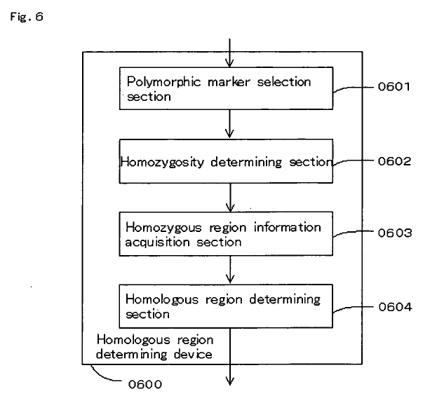

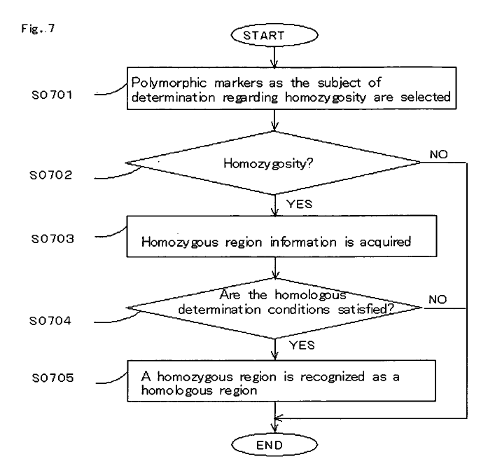

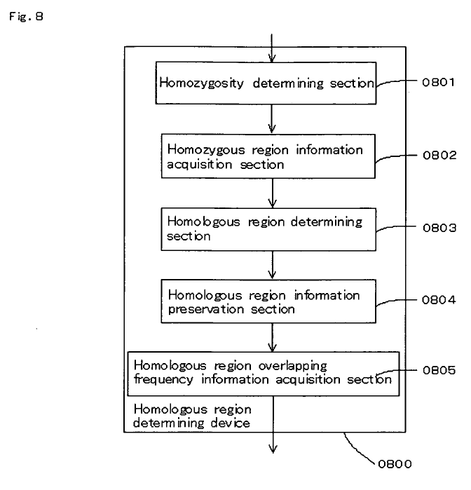

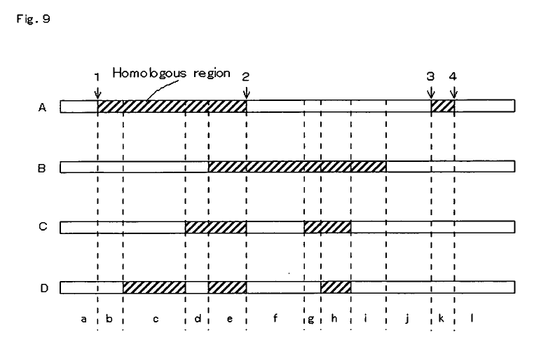

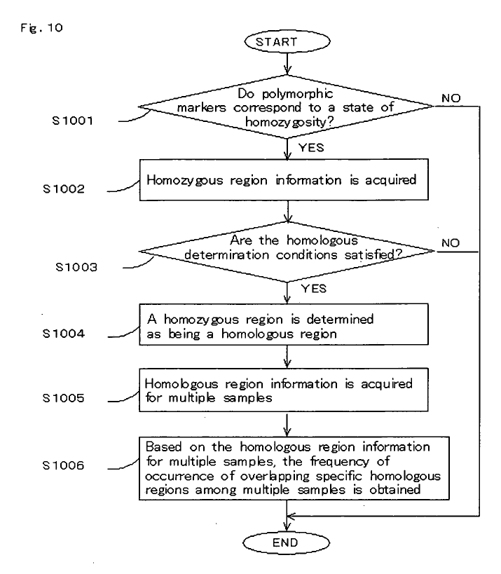

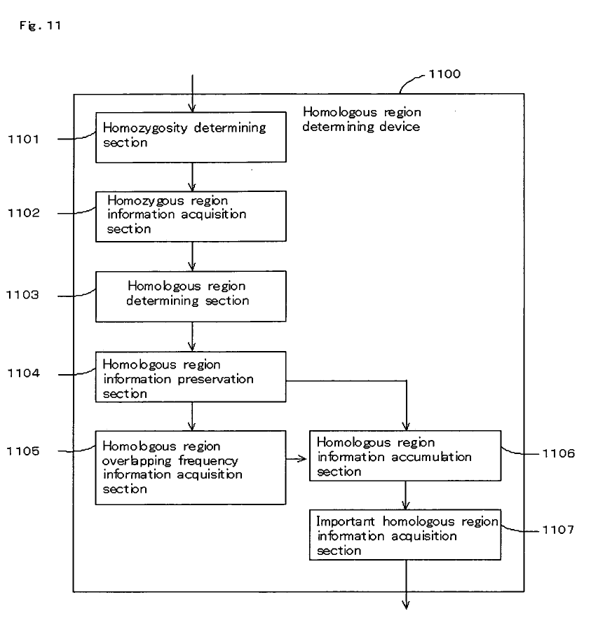

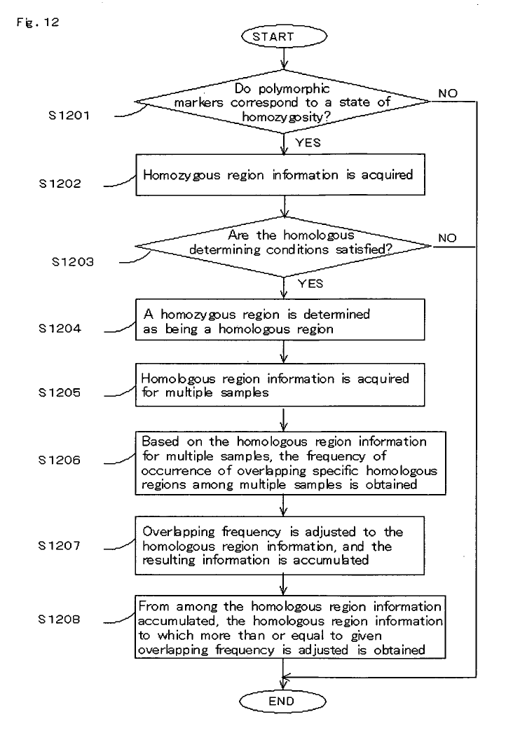

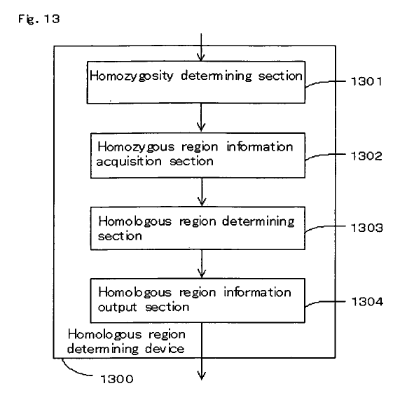

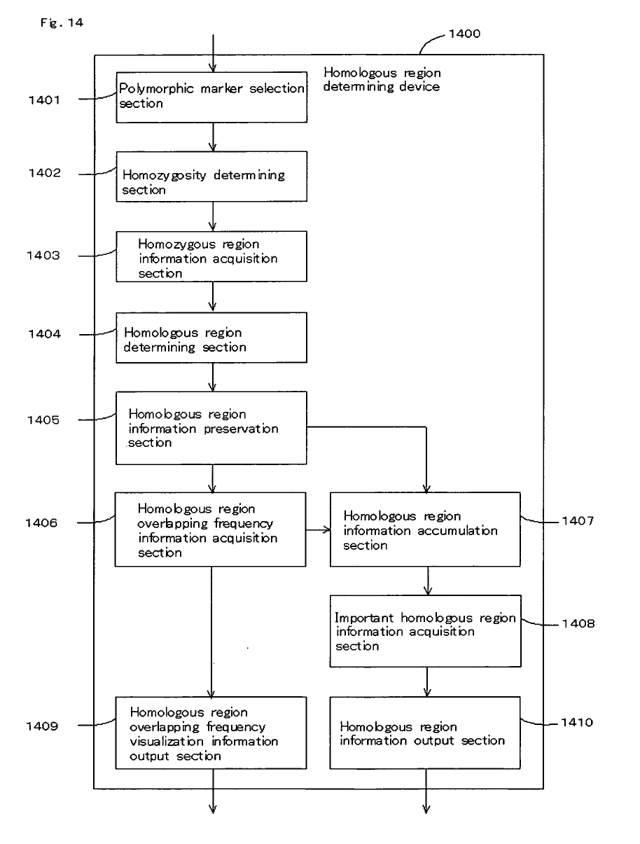

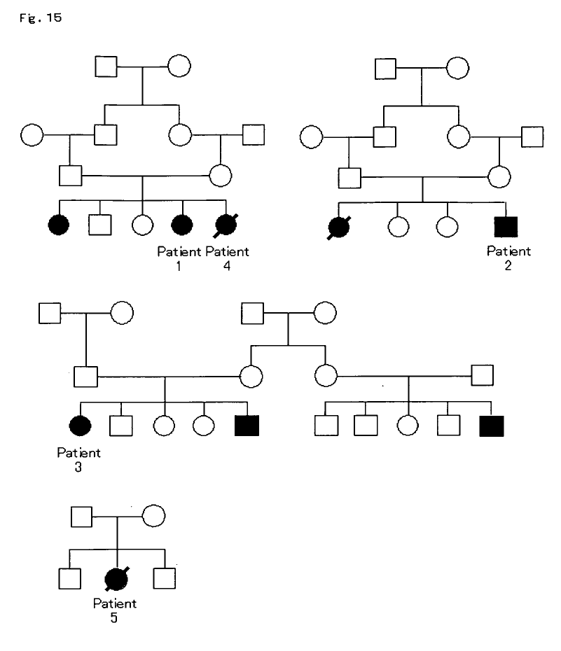

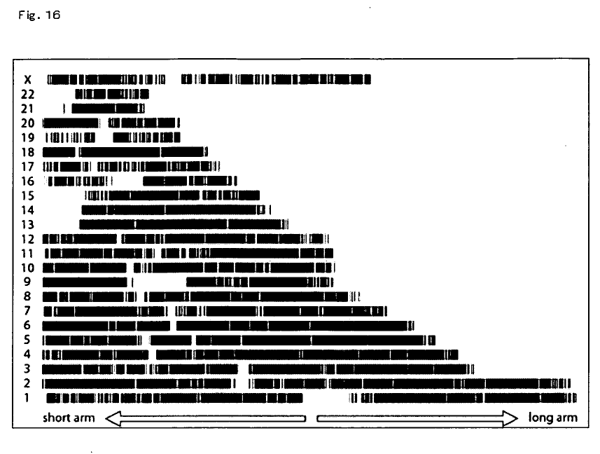

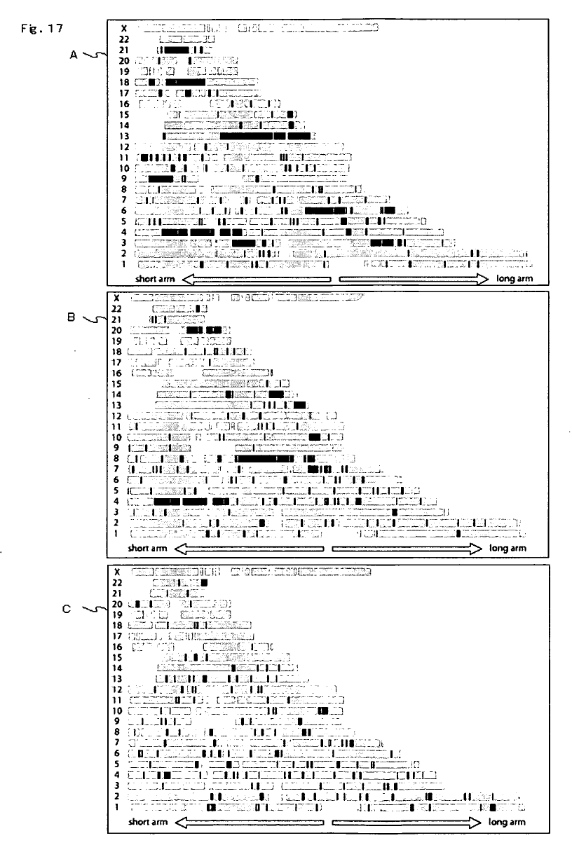

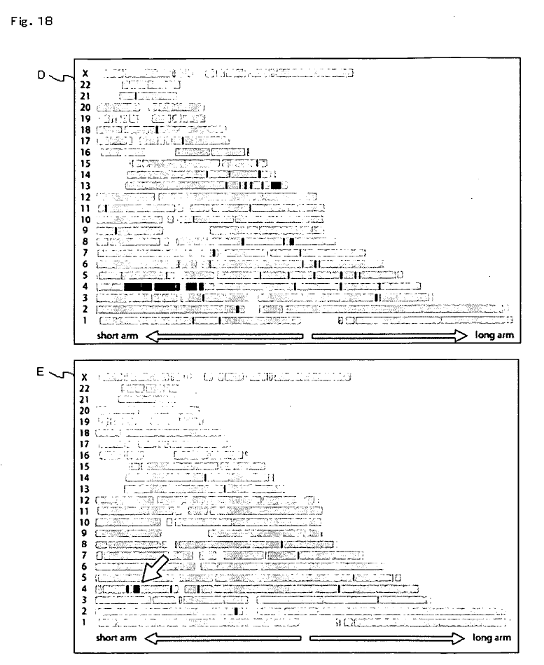

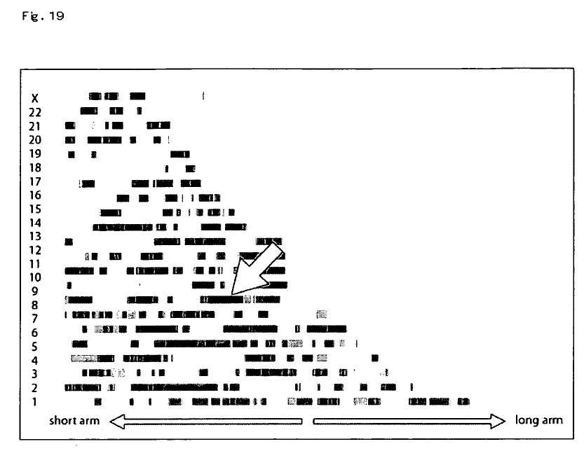

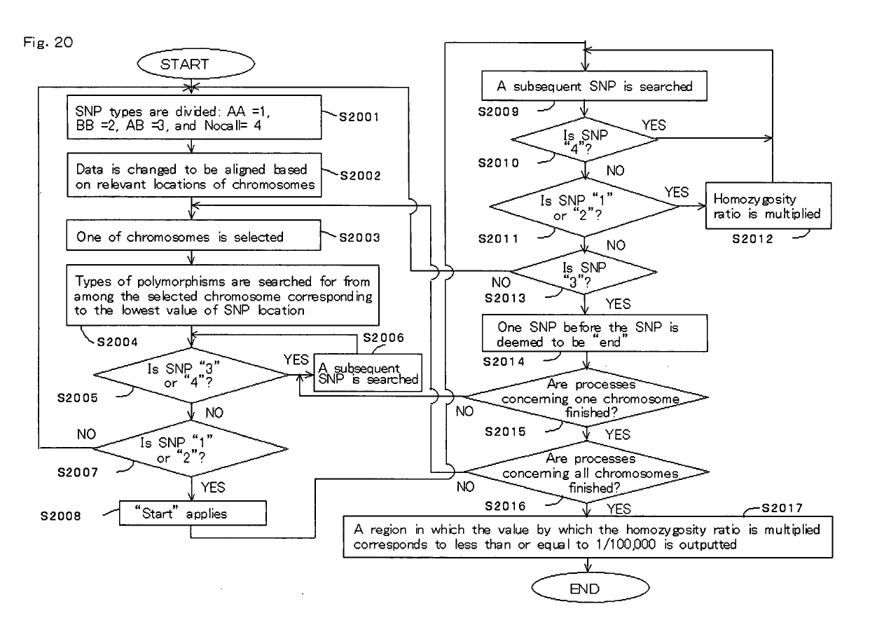

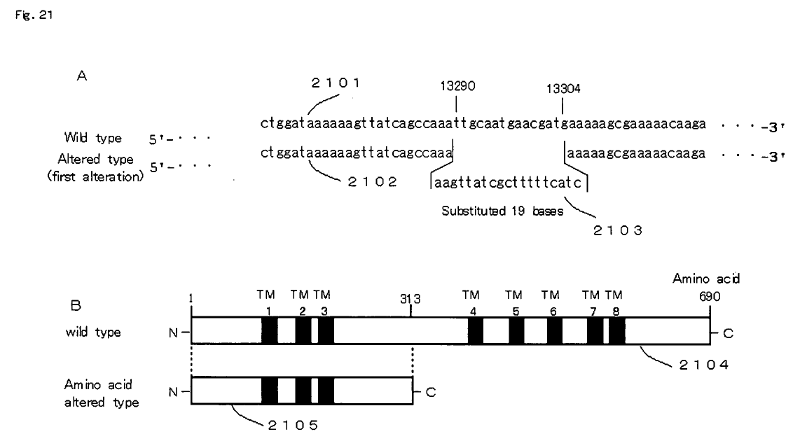

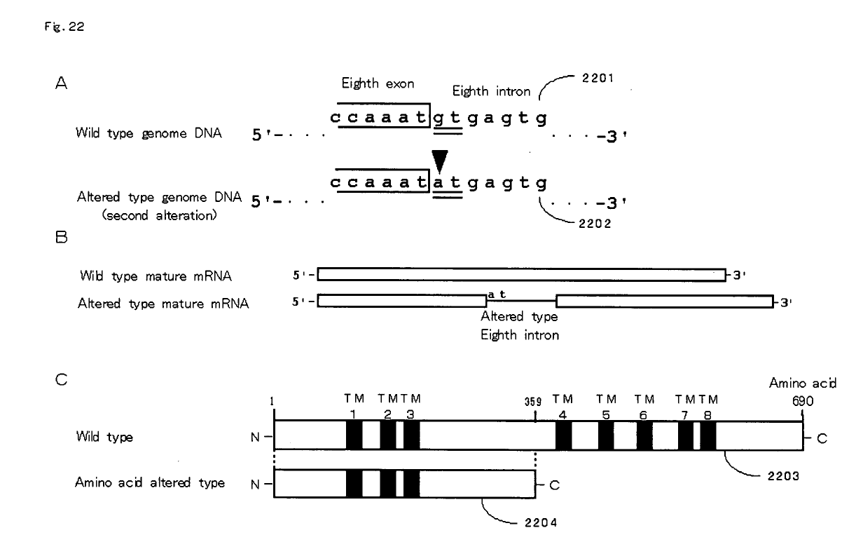

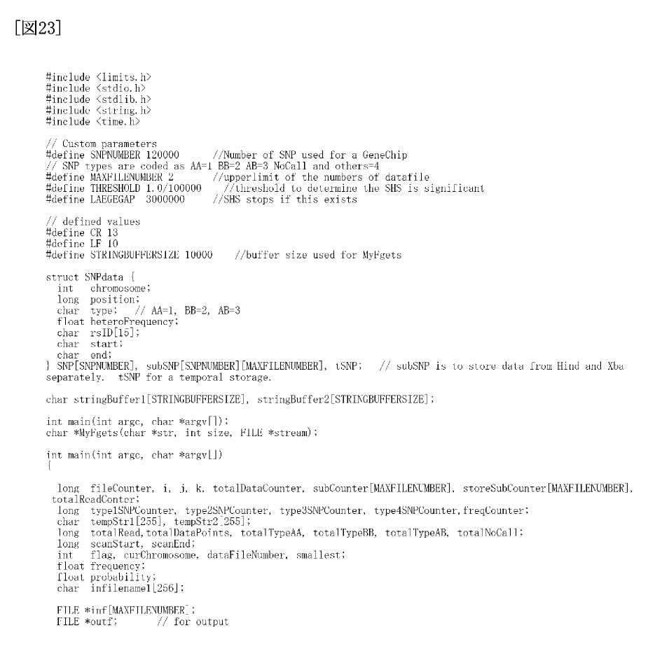

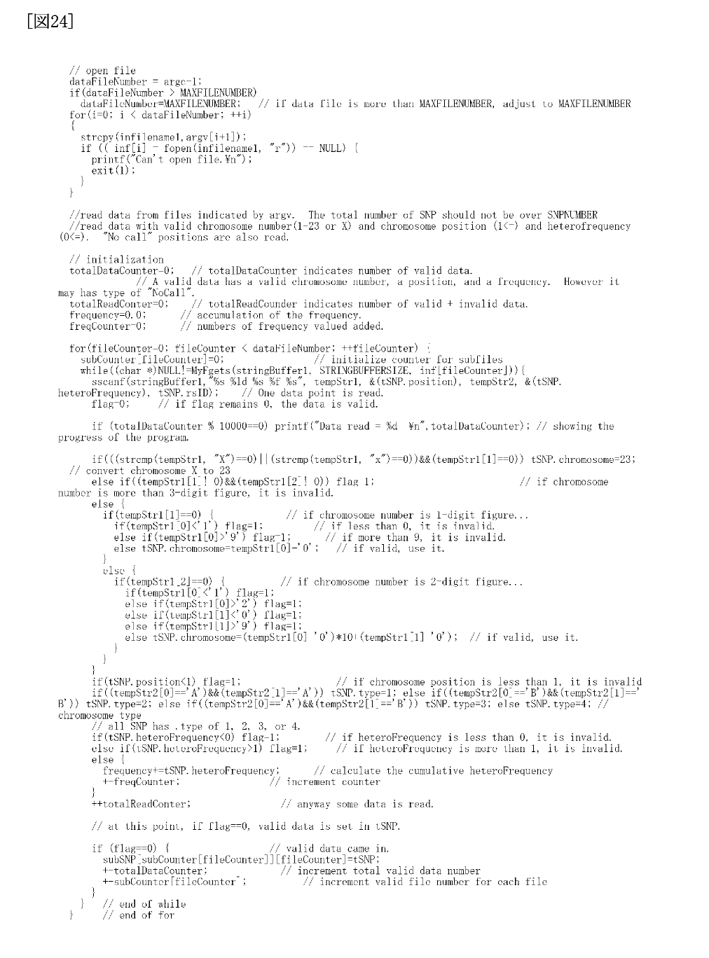

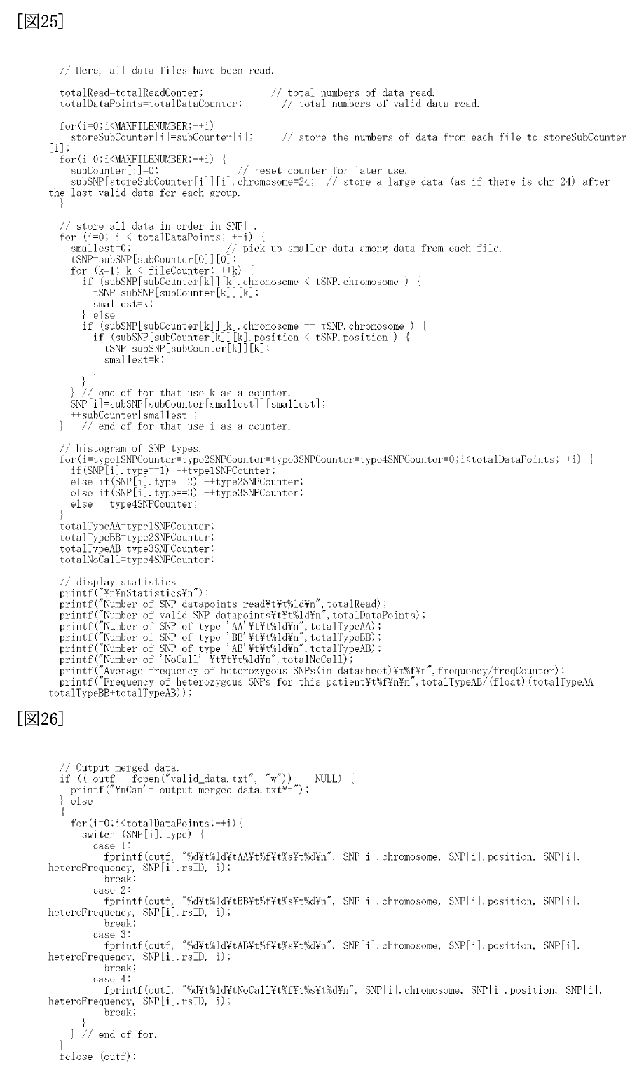

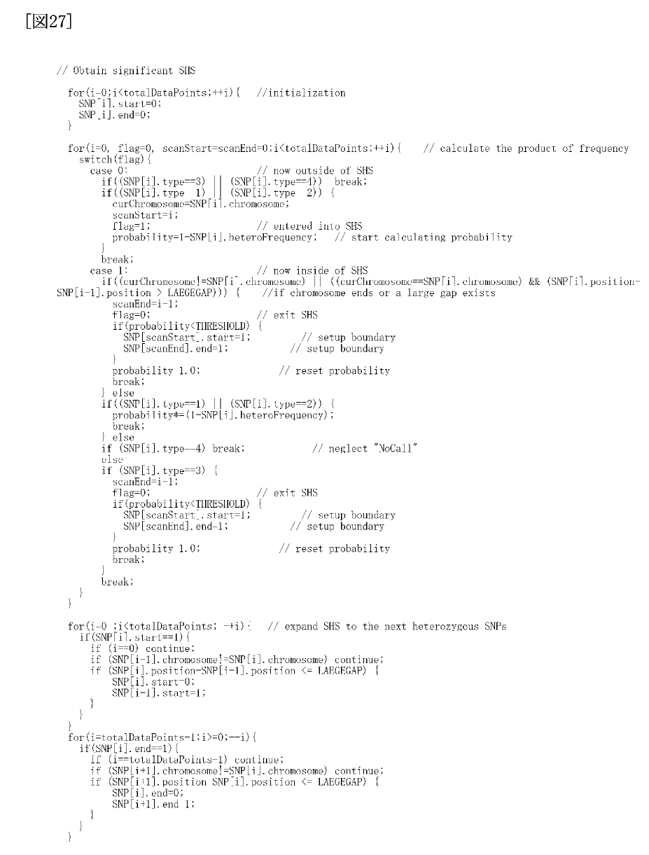

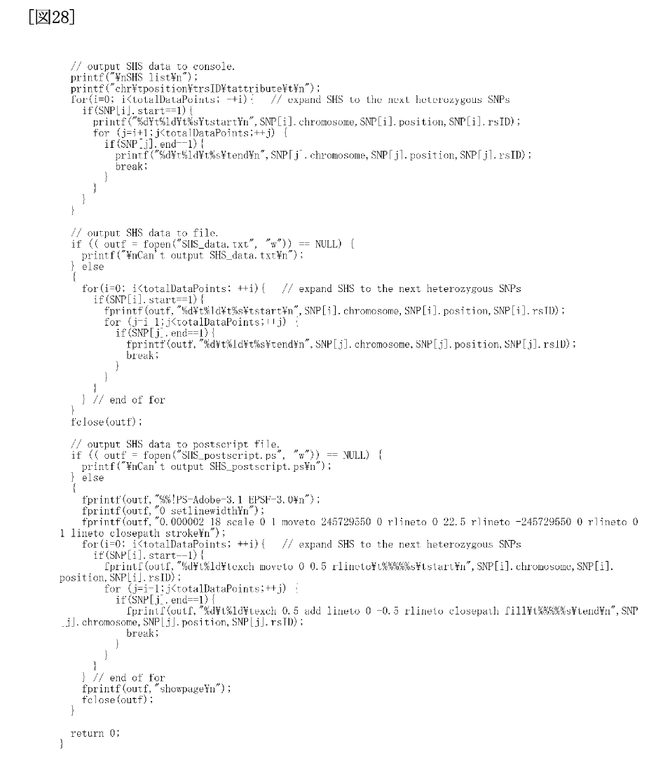

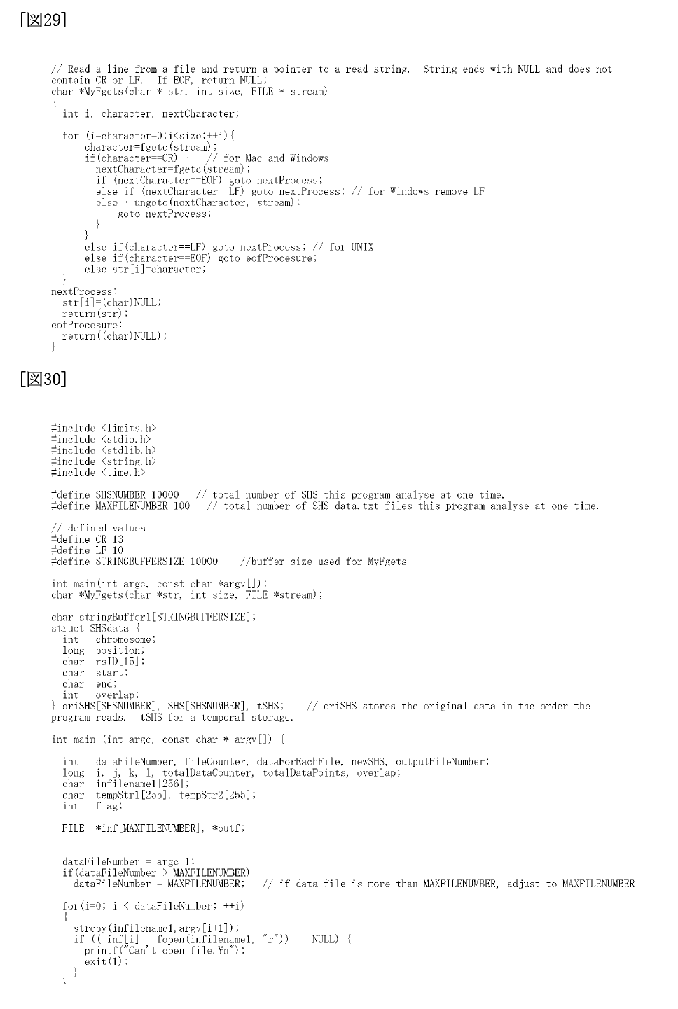

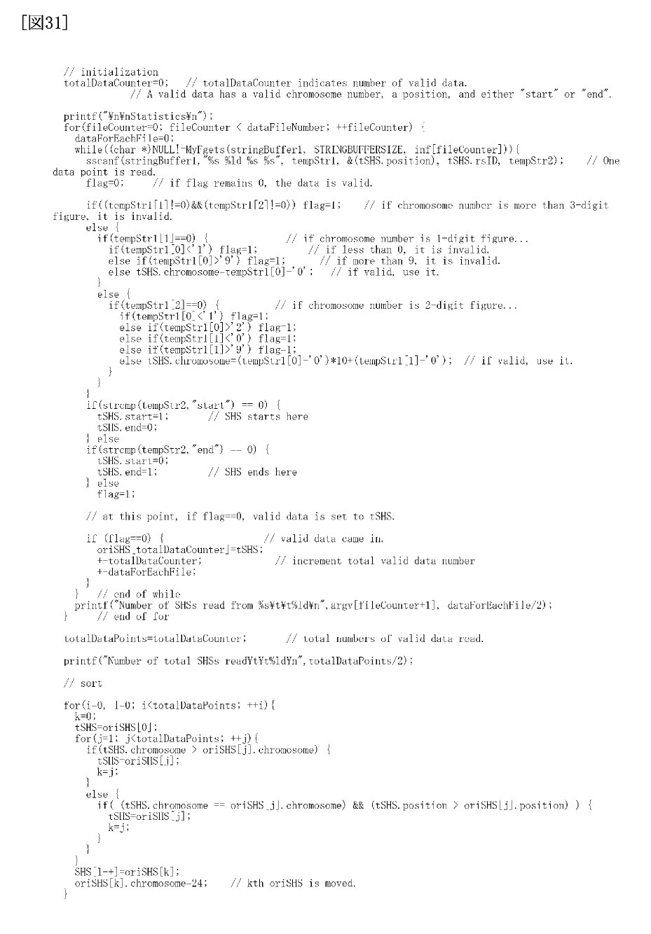

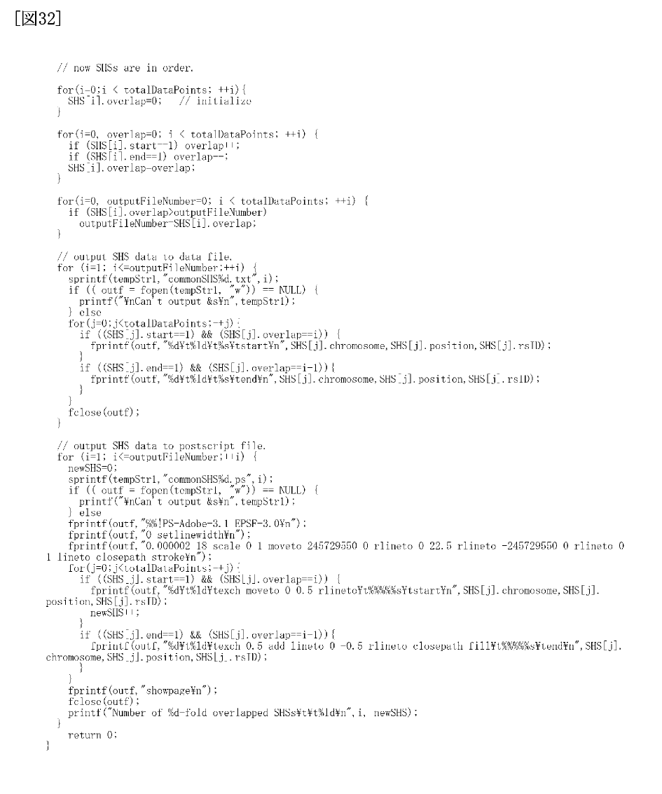

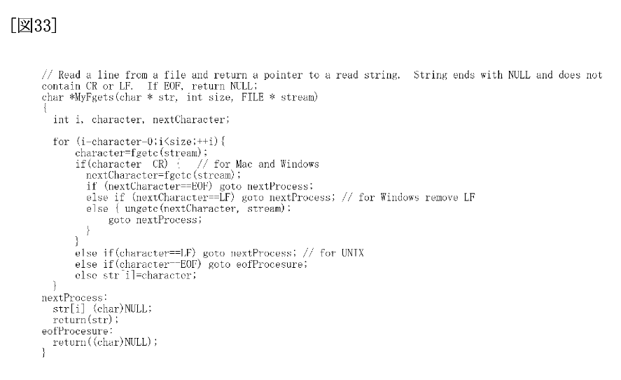

1. A homologous region determining method, comprising the steps of:
determining whether the bases making up polymorphic markers of sample DNA indicating a state of diploidy or polyploidy indicate homozygosity;
acquiring the homozygous region information showing the region of sample DNA in which the polymorphic markers that have been determined as corresponding to a state of homozygosity are contiguous, from among the polymorphic markers that have become the subject of the determination by the homozygosity determining step; and
determining that a homozygous region is a homologous region, when continuous probability and/or continuous distance regarding polymorphic markers included in the homozygous region information satisfy given homologous determination conditions.

2. A homologous region determining method, comprising the steps of:
selecting polymorphic markers as the subject of determination regarding homozygosity selected from among polymorphic markers of sample DNA indicating a state of diploidy or polyploidy;
determining whether the bases making up the polymorphic markers selected by the polymorphic marker selection section indicate homozygosity or not;
acquiring the homozygous region information showing the sample DNA region in which the polymorphic markers that have been determined as corresponding to a state of homozygosity by the homozygosity determining step are contiguous; and
determining that a homozygous region is a homologous region, when continuous probability and/or continuous distance regarding polymorphic markers included in the homozygous region information satisfy given homologous determination conditions.

3. The homologous region determining method of claim 2, wherein the polymorphic marker selection step selects polymorphic markers through all chromosome regions of the sample DNA.

4. The homologous region determining method of claim 2, wherein the polymorphic marker selection step selects polymorphic markers included in regions corresponding to candidate gene regions.

5. The homologous region determining method of claim 1, wherein the sample DNA is of plant origin.

6. The homologous region determining method of claim 1, wherein the sample DNA is of animal origin.

7. The homologous region determining method of claim 1, wherein the sample DNA is of human origin.

8. The homologous region determining method of claim 1, wherein the sample DNA is of Japanese origin.

9. The homologous region determining method of claim 1, wherein the polymorphic markers correspond to SNPs.

10. The homologous region determining method of claim 1, wherein the polymorphic markers correspond to microsatellite polymorphism.

11. The homologous region determining method of claim 1, wherein the polymorphic markers correspond to VNTR polymorphism.

12. The homologous region determining method of claim 1, wherein polymorphic markers are based on a combination of more than two of any of SNP, microsatellite polymorphism, or VNTR polymorphism.

13. The homologous region determining method of claim 9 as the step in which the sample DNA is of human origin and in which 10,000 or more SNPs from all chromosome regions of the sample DNA are selected.

14. The homologous region determining method depending from claim 9 as the step wherein the sample DNA is of human origin and which selects 100,000 or more SNPs in all chromosome regions of the sample DNA.

15. The homologous region determining method claim 1, wherein in regards to homologous determining conditions of the homologous region determining step, the probability of a homozygous region repeating regarding the polymorphic markers shown in the homozygous region information is a value smaller than the probability that selected from a range of 1/10,000,000 to 1/10,000.

16. The homologous region determining method of claim 1, wherein in regards to homologous determining conditions of the homologous region determining step, the probability of a homozygous region repeating regarding the polymorphic markers shown in the homozygous region information is a value smaller than the probability that selected from a range of 1/5,000,000 to 1/50,000.

17. The homologous region determining method of claim 1, wherein in regards to homologous determining conditions of the homologous region determining step, the probability of a homozygous region repeating regarding the polymorphic markers shown in the homozygous region information is a value smaller than the probability that selected from a range of 1/1,000,000 to 1/100,000.

18. The homologous region determining method of claim 1, wherein in regards to homologous determining conditions of the homologous region determining step, the probability of a homozygous region repeating regarding the polymorphic markers shown in the homozygous region information is a value smaller than the probability that selected from a range of 1/1,000,000 to 1/5,000.

19. The homologous region determining method of claim 1, further comprising the steps of acquiring the homologous region information showing a region that has been determined as being a homologous region by the homologous region step in response to multiple samples, and of acquiring the frequency of occurrence of overlapping specific homologous regions among multiple samples obtained based on the homologous region information for multiple samples that has been acquired by the homologous region information acquisition step.

20. A gene screening method, comprising the steps of:
identifying genetic sequences included in the homologous regions as determined by the homologous region determining method of claim 1; and
comparing said identified genetic sequences with sequences of normal genes.

21. A gene screening method, comprising the steps of:
determining whether or not the homologous regions as determined by the homologous region determining method of claim 1 could contain genes that have already been known to function in a homozygous state; and
if an affirmative answer is given in the determining step, comparing sequences of the known genes with corresponding genes of sample DNA.

22. A gene screening method, wherein the sample DNA corresponds to a disease, said gene screening method comprising the steps of:
if the homologous regions as determined by the homologous region determining method of claim 1 contains a gene that is expected to be related to the disease, identifying the sequences of the corresponding genes in the homologous region of the sample DNA; and
comparing the thus identified sequences with sequences of normal genes.

23. A homologous region determining device, comprising:
a homozygosity determining section in which whether or not bases comprising polymorphic markers in sample DNA indicating a state of diploidy or polyploidy indicate homozygosity is determined;
a homozygous region information acquisition section in which from among polymorphic markers as the subject of determination carried out by the homozygosity determining section, polymorphic markers that have been determined as indicating homozygosity acquire homozygous region information showing a sequential sample DNA region; and
a homologous region determining section in which continuous probability and/or continuous distance regarding polymorphic markers included in homozygous region information that will be acquired by the homozygous region information acquisition section satisfy given homologous determination conditions, it is determined that a homozygous region is a homologous region.

24. A homologous region determining device comprising:
a polymorphic marker selection section in which polymorphic markers as the subject of determination regarding homozygosity are selected from among polymorphic markers of sample DNA indicating a state of diploidy or polyploidy;
a homozygosity determining section in which whether the bases making up the polymorphic markers selected by the polymorphic marker selection section indicate homozygosity or not is determined;
a homozygous region information acquisition section in which from among polymorphic markers as the subject of determination carried out by the homozygosity determining section, polymorphic markers that have been determined as indicating homozygosity acquire homozygous region information showing a sequential sample DNA region; and
a homologous region determining section in which when continuous probability and/or continuous distance regarding polymorphic markers included in homozygous region information that will be acquired by the homozygous region information acquisition section satisfy given homologous determination conditions, it is determined that a homozygous region is a homologous region.

25. The homologous region determining device of claim 24, wherein polymorphic markers are selected through all chromosome regions of the sample DNA.

26. The homologous region determining device of claim 24, wherein the polymorphic marker selection step selects polymorphic markers included in regions corresponding to candidate gene regions at the polymorphic marker selection section.

27. The homologous region determining device of claim 23, wherein the sample DNA is of plant origin.

28. The homologous region determining device of claim 23, wherein the sample DNA is of animal origin.

29. The homologous region determining device of claim 23, wherein the sample DNA is wherein the sample DNA is of human origin.

30. The homologous region determining device of claim 23, wherein the polymorphic markers correspond to SNPs.

31. The homologous region determining device of claim 23, wherein the polymorphic markers correspond to SNPs.

32. The homologous region determining device of claim 23, wherein the polymorphic markers correspond to microsatellite polymorphism.

33. The homologous region determining device of claim 23, wherein the polymorphic markers correspond to VNTR polymorphism.

34. The homologous region determining device of claim 23, wherein polymorphic markers are based on a combination of more than two of any of SNP, microsatellite polymorphism, or VNTR polymorphism.

35. The homologous region determining device of claim 31 in which the sample DNA is of human origin and in which 10,000 or more SNPs from all chromosome regions of the sample DNA are selected at the polymorphic marker selection section.

36. The homologous region determining device of claim 31 in which the sample DNA is of human origin and which selects 100,000 or more SNPs in all chromosome regions of the sample DNA at the polymorphic marker selection section.

37. The homologous region determining device of claim 23 in which in regards to homologous determining conditions, the probability of a homozygous region repeating regarding the polymorphic markers shown in the homozygous region information is a value smaller than the probability that selected from a range of 1/10,000,000 to 1/10,000 at the homologous region determining section.

38. The homologous region determining device of claim 23 in which in regards to homologous determining conditions, the probability of a homozygous region repeating regarding the polymorphic markers shown in the homozygous region information is a value smaller than the probability that selected from a range of 1/5,000,000 to 1/50,000 at the homologous region determining section.

39. The homologous region determining device of claim 23 in which in regards to homologous determining conditions, the probability of a homozygous region repeating regarding the polymorphic markers show in the homozygous region information is a value smaller than that selected from a range of 1/1,000,000 to 1/100,000 at the homologous region determining section.

40. The homologous region determining device of claim 23 in which in regards to homologous determining conditions, the probability of a homozygous region repeating regarding the polymorphic markers show in the homozygous region information is a value smaller than the probability that selected from a range of 1/1,000,000 to 1/5,000 at the homologous region determining section.

41. The homologous region determining device of claim 23 in which the homologous region information as information showing the homozygous region determined to satisfy the homologous determination conditions by the homologous region determining section is visualized and outputted at the homologous region determining section.

42. The homologous region determining device of claim 23, further comprising:
a homologous region information preservation section in which multiple pieces of the homologous region information showing a region that has been determined as being a homologous region by the homologous region determining section are preserved in response to multiple samples; and
a homologous region overlapping frequency information acquisition section in which the homologous region overlapping frequency information showing the overlapping frequency among multiple samples in regards to specific homologous regions is acquired based on homologous region information for multiple samples preserved by the homologous region information preservation section.

43. The homologous region determining device of claim 42, further comprising a the homologous region overlapping frequency visualization information output section in which the homologous region overlapping frequency visualization information corresponds to visualized and outputted homologous region overlapping frequency information obtained by the homologous region overlapping frequency information acquisition section.

44. The homologous region determining device of claim 42, further comprising:
a homologous region information accumulation section in which the overlapping frequency obtained through the homologous region overlapping frequency acquisition section is adjusted to the homologous region information, and that the resulting information is accumulated; and
an important homologous region information acquisition section in which from among the homologous region information accumulated in the homologous region information accumulation section, the homologous region information associated with a frequency that is greater than or equal to a given overlapping frequency is acquired.

45. The homologous region determining device of claim 44, further comprising a homologous region information output section in which the homologous region information to which more than or equal to given overlapping frequency is adjusted and such information is obtained by the important homologous region information acquisition section is visualized and outputted.

46. A gene screening method in which genetic sequences included in the homologous regions determined by the homologous region determining device of claim 23 is identified and is compared with sequences of normal genes.

47. A gene screening method in which the homologous regions identified by the homologous region determining devices of device of claim 23 is overlapped with the homologous region for which information is accumulated in the homologous region information accumulation section, and the gene sequences included in the overlapping region are identified and compared with the sequences of normal genes.

48. A gene screening method in which it is determined whether or not the homologous regions determined by the homologous region determining device of claim 23 could contain genes that have already been known to function in a homozygous state, and in the case of a region that could contain a gene that has been already known, sequences of corresponding known genes and corresponding genes of sample DNA are compared.

49. A gene screening method in which in case that the sample DNA corresponds to a disease, if the homologous regions determined by the homologous region determining device of claim 23 contains a gene that is expected to be related to a corresponding disease, the sequences of the corresponding genes in the homologous region of the sample DNA are identified and compared with normal genes.


SEQUENCE LISTING (10 sequences)

SEQ ID NO: 1 - DNA, 21748 bp, Homo sapiens
  mRNA for gene SLC34A2 at positions 6682 to 6793, 6886 to 7023, 8383 to 8511, 10309 to 10452, 12061 to 12368, 13265 to 13360, 14184 to 14304, 15670 to 15837, 16879 to 16995, 17088 to 17212, and 18718 to 19329
ccatatatacccggggcgctgcgctccacctggccgccgcctccagcccagcacctgcggagggagcgctggtgagtaccgccgccggggcaggggcgcttcctcgctctttcaaggactttgattcacttaattcttgcaaatacctctcggtgctgacttcaaggaacttggctggctttgggccgcagaagtgaaaaacacaaagctctccacaatgttcaagttgttttcttcttaatgttacggttattgcttttattacagcttttgctgctacactcttacgatcgacagtgttattaatcagccacacttgtggattctaaaatgatcactgttttgagagtcgtggtttggaagaggaaaggcccaggggatagaaatctccacctaggaatcaggatagctaggctctttgtggctgggtcaccaactagccatgtgacaacgctagtccctgtgagcccctgggctgccgtcttccattctttataagaaggggaatgcgacatctcaggaccatgaaaattctaggtttgtggtggctgcctggtgatgtgacactgccccccacccaagtgtgacttccacgtggtacagtgcttgtttgcagttatttaaggtgcctaggagacagtcttagtttgtttctattagggcccgtggccatcagcgaagggtccgtccttcagccgtcttggggagcaaagcccgcaatttatgttttccaagccacaaatgggtgagcaggctggggagataacttacaaggagatgtgcacaggagtggttgggttcggggactgcgcatcctcaggccagaaagttgggggctgcatggcatggagttagttcaggttccctgcaacggtgcctgccaccggtggctcgcactgcgctgaagagagttggaatctcgttttctttgaggaggtccctttttcaaacatgttgatttcatggaagcagcttgtggatttcatcactcgagtcccctcttcctgtttgggtgccccagggggtttctgtgttagccctgatcgctctgggccttgtcgttctttcctttttccttcttcccttgtgtgctggcagttctttaacttgtcctgggttgggtattcttggaaaactgtgcttgtaggagtggccgttgtttcagattgttaaagaaaaacaaaaataaaaaagattgcctcttttgtcccatgcccgctgatcatgatcttctctgcttttgtaatttggggaatgggtgataagaaaggcgtgctttgtaaaaggtttggatgctaaacaaattgccccatccaaaactccccaaattgctcaactgtctcaatgaagcaagtcccctgtggtatgaggatgggcttctgattgttgctgttagacacaaaggggacaaaaaaatagcccgaattcaatgcaatgaggaaaacagcctcacagaatcagagtgctttggaataaactgggctttagtaaccagtttcaggcatcttccactcctccatcctcacccacacccccacttccattctggggaccgcagggagcaatcaggacatcccagggaatttattcatctcagtgaatttttaaaatgcaaaggtacaaaaagagggctataaaaagattgcggtatttttaagatgtttatatttacaaatttaactgctttaacctccccccccaatacaatgcagcacaactgatatagttacaagttatttacaggatttattttaagatatcataaatacaaattgatagtgcatggtgagggtgtggtttggtgatatggcccaagtttaaagtttattagtgtgccacatcaaaaatgtaaactcaagttatctatcaatgtgaggcccaggccaattgcttccttgactcctctcatcactaccaccattgtgtccttgaagagattattattagcagtaatgtagggacagggtctcactatgttgtctaagctggtctcaaactcacaggttcaagggactctcccactttggcctcccaaagtgctgggattataggtgtgagccaccacccttagcctataggggtgattatttttccttgtgatggctgtaagggaagcagaggaaaaggagacgtcaccctcttgggaatttttgaagcacagagtcattctttcaaagtacaagaaagttttcctgggcatcccgagaacactggcctgcatgaggttgaaattatctctgcagtgttgtggtgcctatgtgatttgaagccattaacctctctgtggctcagtttttccctctatagaatgaggatactgttcattttaaaggagactctaagtttacatagatacatgaaatgctgtgagtaaatttgtgcaccaatttgcaagtgccaggcacaaagtctttaataaacactacttggctaccatgtgcccagcactgtcctatgtgctggggatacaatggccatactctagtccctgtgcttgtgaaggctgccattctgatgggagactgtgtgtccatgttctggttcctcacaggaccactgccctgggcttcctcctctccccatttcttcagatggccttgactgctgctctgggcacaaggggcccaggaacaggtcttggttggagaagcccaaacaatgcgaatgtaggggttgggtgactgccaggtaccctacattcccctgcttcccaagtctgagccaagtcaagaaagttcctctcctgggtcttcaagaaagaataagagctctttctggcagtgggtgcccatgtggtgcaggaagtggagccaagagggtgaggcacacaggcaactcggttatagaggccagtgtggcagcgggggagcccctccctgggctttagtaaaaagcttccaaatgatgacttctcccttcctctgctgcttctggcaggtggcaagaatgtgctggagtgaggccttgggaaggctgggagagaacaaggcagtggttgcgccagggctatgggggtaggagaaaggcttagaggggctcagagctccagggtacattctctgcctgcaccatttaatacccacccccacccacccaccccacccccaccaccctcccctcctgctcatccattgcaacattcttgggcgggcatttccagcatctgaatgatcatgtgggagtgacctgcagacatgccaatctagccagctatccgaaaacactcagagccctcaatgtgcctagacctaaggcccctgggtgccctggtgctttgagggctggagaatttctcctccctttgcccctggctgtgagatctgtggaaaagcttctggctttggagccagcagtcttgggttcatatcctgacaccagctccaggtagttggaggcaagcttctttgtttctctgtgcctcagtttctttgtctgtacactgagaagaaaaactccttccttggtagatttaatgaggtcacaaactgtataggacactcctctgccacaaaacaaacattagcatctttctctcacaggctttggattcatttattcaaatattaactgagatccactaccatgctgagcattggagatgaagagcttatagtctagggtacacacagtggaaatgaggccatttactgttcatgacacagtgcatttccacgtaataaacactcttgtttattgttcatgacacagtgcatttccacattgtgtcatgaacaataaacaagagttccgagtgtttactctgccaggcaccatgctctagcctcacctacacttattaaccaattagtcctcataacagctcagtaagcaagatggtattgtccccatttttacaatgatgaagctgaagctcagagtggctcagggttgcatatagtgaggggcaggcctctgcagagccagccctccctcactgtgattcgcctctctccaggtatcactgagccatcttacctttgtgagcttagaggttgagttgcatcttttaaaataattttcttctaaaatttaaaaaatcacccacagtctcaccactcccttttacctccagagaacatttctggatcttgtctgccttgttgagatgatgctgtgaatgggaaaaagaagtgctttagatgtgaagtttatctaaagcatttccctatggttttactgattcagtataacctctttaatggcatgctcttacaatgcatggacataccatagttagaaggagtaatttctcttttttcattttttcacactgtaaatgatagagcagcaaatatctttttggtctacatcacattcattctatcataaatatgcccggcagggctcaagaccttaaagatgaagtgatggacaagacaaagtccactttttggtagggtttgcagtctggtaagcaggaaataggttgacgaagaaaggagtcaggtggactcaggctctaagaagccctcttggaagaggtgatcagagtaagttaggtatttctgaatttctgagaggaaatattcctagcttcctgattcctagaagtgaaatgactgggtcacagacgtgtgatgaacataaggcactttggacagtttgctaacttttccagagcttctagcagtgttcaactcataacatcttctccagcatagcataatgaacttaaattctttttgctactttgatagttgtcactgttttagtttgaatatatgggattgcttctgtttttttgagacggagtttcactctttttgcccaggctggggtgcaatgatgtgatctcagctcactgcaacctccacctcccagattcaagtgattctcctgcctcagcctcccgagtagctggaattacaggtgcatgccaccacgcctggctaattttgtatttttagtagagacggggtttcaccatgttgtccaggctggtctctaactcctgacctcagatgatccacctgcctcggcttcccaaagtgttgggtttacaggcatgagccactgtgcccggcctggattgcttcttttttaataggcattttagccatttgttccttttctgtgaattgcctttctccaatcttttggactgctttgtctaatggacaaggactaattgtctaatgtcaaagaacaaggactgcaaggactggaatgtgaggtgtgtcaagtaccatgtaactgagaaaggccttgtgaagaaaatagattgtaagaaggtcttttctgaaggctgaaaagaatgtggttctaacaaaggatgattaatccagacagcatgacatagtaataattgctgctatttattggatgcctcctcaatgccagtcactttctagacttttaatttcactattagtctcataaccctgcaagaactgttccattttatagatggagaaactgacactttatgctcagaggcataaagggacttgcctaaatacacactcattaataggtggcactgctggattttaaaaccaggtccatcactcttttcacagaaggcctgggtggtaggtaaaacacgcagaaggtatatgaacacctgcatatacttatatattttgagacagagttttgctctgtatcccccaggctggaatacagtggcatgatctcagttcactacaacctccgcctcccaggttcaagcaattctcatgcctcagcctcctaagtagctgggattacaggcacccaccaccatcccctgctaatttttgtatttttagtagagacagggttttgacacgttgcccaggctggtctcgaacttctgacctcaggtgacccacccaccttggcctcctaaggtgttggagttacaggcctgagccacctcgcctggcccctgcatatatattcacatacacactaagttgtataaatctgttaaattccactggataaggttttgcatagatttgcagtgcacttgtcttgatataagaagtagatggggttccattgaagggttgtgaggatttggcatgctatgagacaacaaagatgaatgggcttgcaccctgcctttttctttttttttttttttgagaaggagtttcacttttgtcacccaggctggagtgcaatggcacaatctcagctcactgcaacctctgcctcccaggttcaagcgattctcctgtctcagcctcccgagtagctgggactataggcgtgtgccaccacgccggctaatttttatatttttagtagagatggggtttcaccatgttgtcaaggctggtctcaaactcctgacctcaggtgatccacctgtcttggcctcccaaagtgctgggattacaggcgtgagccacggtgcccgttgcaccctgcttttgactgaaggagagagtcaagacaaaaacaagtttacgagattaaaggtggtacatgcagctaactcaacactgtacgaggtagtttgctttagcgactgggtgagtgtttgaaggggaaccacagaggaaatatggtgcttaatatgccacacttagggggcataagtgtgaaatctttcccttccttttactgccgcctccggccgaaacccccacccagttgatgctttgcaaccaatggttcttcctcttatagcatctcggtgtgcctcctttccatgactgctgctttaagctgttttctcatccacagaccatggctccctggcctgaattgggagatgcccagcccaaccccgataagtacctcgaaggggccgcaggtcagcagcccactgcccctgataaaagcaaagagaccaacaaaagtaagtgtcgctcgtttgtctgcagatcggcctttgtgaggaccccaggagactcaggtctgattcctcattaccccttttgcttgtttcagcagataacactgaggcacctgtaaccaagattgaacttctgccgtcctactccacggctacactgatagatgagcccactgaggtggatgacccctggaacctacccactcttcaggactcggggatcaagtggtcaggtaaaagtgaggccagctgagacattcaggagggaaacttcctgtggttcacggtgtcatcatcctcctgtccctcccccttttcattgttctgattcccttgaagcaagggtcctggctagggatgtcaccctctcatctttttttttttagacggagtcttgctctgtcacccaggctggagtgcagtggtgcgatattggctcactgcaagctctgccccgtgggttcaagcagttatcctgcctcagcctcctgagtagctgggattacaggctaatttttgtatttttaatagagatggggtttcaccatgttggccaggctggtctcgaactcctgacctcgtgatccaccctcctcgaccttccaaagtgctgagattacaggcatgagccacagcacccggccaccctcccatctttgaggtagagtcaaagcatgttggagtaattgggggtcattcattcaacaaatgtgccaggcactgtggttatagcaaagatggacattgtctggctccttataattcgctgtgggtgatgaggttccaggtactgctttggagatgctgtggatacagtgatgaacagaaaaggcccctcttctcagagagctttcagaggggtcccctcaccaaaatgtgggcatatttctagtaaaagaaaatatgcagccagacagctggatgcaagcaatcactgtttcatttccaaatgtatgtagggcagaaggtctgtctgtgaactagacagcaaggaggaaaaaggagattggggtgtggggtggggaggagaggtaggaaggaaagagctgggagggggaagatgagaaccattttaaaagccagtggagtaaacagcacttaaggatgtagcatcccaggatgtatgatgccccaagccctccataaattgaggctgattgctgctgtgcagctgcattctagaaaatactctgtgctgtttggaacgtgacagccattatctccgaccctgcacttagcaggtggctagtgctgtcaactgcctcactcagtgacactgtggctcagctgagcatggagcctggtttttctgtcgcagaccacattgaacccctcctcccaaacgcaaatcctttagaggcactttaccaggggtttcagctaaatggaccacagcggtaactgctttgaaagctgcagcgatggctgcttcccatctgtaaggctggctagacataagaacttaacggctgccaggctgggaagggaggggaggtcgggggagctctgagctcattgccaaacttctcagggtttccaacactaaaagtttcatgcctttctctctctccccccatcccacccccctgcagagagagacaccaaagggaagattctctgtttcttccaagggattgggagattgattttacttctcggatttctctactttttcgtgtgctccctggatattcttagtagcgccttccagctggttggaggtaagaatgaaagggtgagaggtctgcgggtgaggggcattatcttgaaatgtggttccgagagtaaaactcagcaagccctctccagctgcagcctcctggagtgttttaggactggaaccgacctcagatcatttatgaagtctatgctttccgttatagaagagaaaactgaggcctatacaaagggctatgacttggcccaggtggcttaggccaggagctggggctagactattccaagctatccgccagttgagtttgtccacaaaccccaggcaggaagttgtctgacatacagacttagctgaggaattcctatatcctccacaccagagagaattctggaatgagaaagagtgccctttcaaatatgggcccttgctgggggaaggacagggccccttctctgggctggaggaatctgcatccattgtacttaccttcacagcccttagggctgcgtgtgcccatgcagtgtgagaacccaggagtgaagtcaccatgtgcttggttccctttgtacctcaatggtgatgtcagaaacaaacaagcagcactccgtctactgggtgcaggcattttcgggggccctctatcaaacattctttcccagagccctcccatttacggagagatcatgaaccagccctgagactcacagcctgtgattagcagagtaagattcagtctcagatctgggtgacacaaaggaccatggatttctgcaacccttggtgcctttcttgggaacccatctgtgtgacttgggagagttggggaggtgggcattcatgggagagcatgaggggcaaacacttcctggacattgcttttttttttttttttttttgagacagagtttcactcttgttgcccgggctggagtgcattggcgtgatctcagctcaccgcgacctccacctcccggcttcaagcgattcttctgcctcagcctcctgagtagctgggattacaggcatgcaccaccatgcctggctaatttttgtatttttagtagagacggggtttctccgtgttggtcaggctggtctgcaactactgacctcagttgttccgcccgtcctccgcctcccgaagtgctgggattacaggtgtgagccaccgtgcctggcctcctgaacattgcttctgggctccctggtccatgggagcatcagctaggggctttgctgtctggttatctcagacaaatcacctgcatcctctgggcttccaccttctttatacaatggacttgggaatgcattgatggtctcaggtacactcttgagaataactctcccagcattcaattacaggctggtgctagggctaagggtggatagagagatctactcttggaggataaatctgaattgggtatacaccccagagacatccaggaggctcattggaacccactggtcccagtatggagttgaggctgggttcctgagcacagaagggcttgctgactgaggagtgtttgaagagtgggaggatgcagggtgaaggaacaaaagtaaccaggggctcacagtgggtcgggaaccagggcagagagacagagacaggaggcacgtgtgggcagctagtagatttcctgccttatcggggcagcactgggaagaggcatggggaagcaagattccttgggtgcctgcagcgatggaggctggactctgcaacccacagccagctggccttggatggagacttctgtttactcagtgcccacctaatccccctcgatcacgttgtgattgtttttgtttgtttgtttgtttttcccaggaaaaatggcaggacagttcttcagcaacagctctattatgtccaaccctttgttggggctggtgatcggggtgctggtgaccgtcttggtgcagagctccagcacctcaacgtccatcgttgtcagcatggtgtcctcttcatgtgagtcggggcacccatgagcccacctgcattccagacactctcctgtctatctgagggtgggaaggacgggggaggaattcactctgaatatgtccaggccttgccaccattgtcttggtatcttgccccagctacaatgtgtttccctcttgatccaaggcaacttcctgtttccatttcgatggcaggatctggaaatagaccctgctgctggagttctcagctctgaattctctagtactgtactttccagacctgtagccactagccacatgtggcagtttaattttgttgaaccttaattgatttaaaattaaaatataacattcagttcctcagttgtactagttacatttcaagtgttaaagagccacatgtggctggctggcagctaccctattgagaagtagagacatagaacatttccttcactgcagaaagttagttctctgggacagggccactctcagtgctaagaagaaagacaacttaatcacatcacaaattgctctgggacaaccgagcttgggagatggcacactaaacttgtacctgtctgataattgctgcagctacgttggacagaccctctgagggggaccagtttgaactgattattattatttttttttctattttctttttaaatagagacttgggtctcactgtgttgcccaggctggtctcaaacttctgggctcaagtgatcctcccaaaatgctgggattacaggcctgagccacagcccctgaccagaactatttttcttcccaagctagcatcttagccaaaattaactagaatgtttctcgtccagttagaaaaaggtattacactttcaaaatattttacttgtttatatcaaaaaaggtgcagcaatatgctgattttgttggaaaaagttacactgcctagaattaaatgtctgatatccagcacagaaatgatgcttcctggctgggcctagtggctcacgcctataatcccagcactctgggaggctgaggtgggaggctcacctgaggttgggaatctgagaccagcctgaccaacacggtgaaaccccgtctctactaaaaatacaaaaattagctggatgtggtggtgcatgcctccagtctcagctactcaggaggctgaggcaagagaatcactttaacctgggaggtagaggttgcagtgagctgagatcgagccactgctctccagcctgggagacagagcgagatccatctcaaaaaataaaaataaaaaagaaaagaaaaaaaactgatgcttccttaaaggcgagcctgtgtgccatgcacagctgactcaactgtacatgcaagccctgcactcatccacctaagtccttaatcagtgggtgcctcctggtctccttcaggctagccttggatctgcaatagggaggaaggaagaagctagaaaggcactttcttcagattcatagtgaggtgcagagtgaggtgcaaaaaaaaagtttaagaattcacttgcatagaggataaaaactacttctttagaggataccagcataggtaactttagcctgcctccaggctgcctttctaagcttgctaatggtacttttccatcctctagtgctcactgttcgggctgccatccccattatcatgggggccaacattggaacgtcaatcaccaacactattgttgcgctcatgcaggtgggagatcggagtgagttcagaagagcttttgcaggagccactgtccatgacttcttcaactggctgtccctgttggtgctcttgcccgtggaggtggccacccattacctcgagatcataacccagcttatagtggagagcttccacttcaagaatggagaagatgccccagatcttctgaaagtcatcactaagcccttcacaaagctcattgtccaggtaacttagctccttcagagagagaaggagactaacttcctacccacaacatcccctacctgagctgacagatatagagtgtctacaacactattctgggcctggcatggtggctcacccttgttttcccagcactttgggaggcccaggcaggtggattgcttgaggtcaggaatctgagaccagcctggccaacatggcataaccctgtctatactaaaatgcaaaacattagctgggcatggtggcgtgcacctgtagtcccagctatgtgggggctgagaaaggaggatcgcttgaaccccgggaggtcaaggctgcagtgagctgagaattgtgccattgcactccagcctgggtgacaaaagtgagactgtttttaaaaaataaatagatgaataaacactactctgtagctgcctctggaaaaggccaagggaggcagaacttttgggccacctgatagagtgactgaccttgatggttactttgacttccataacatgccacctttattggggtggaggtgcttcttaagactggacacttgagaccactttgttcccactgctgtcctcaacaccagtggacttgataaagtatgatctaccctttcctgcctagagtcatctcagccccttctccctgggtccttgctaggaccttgtgttttcactcttactggttttcttggccattcagtcatccagaaaatatctactgtctgctccctagatatcagtgcctctgcagatagagtgagtcccactttgcctctctgggggctcacagtcacatttatctccttagagcccctctcacttcaaccccctgggtttgtgtcctaaatcagttctgaggatatgctgatggtttcctgtctactgtttccacagctggataaaaaagttatcagccaaattgcaatgaacgatgaaaaagcgaaaaacaagagtcttgtcaagatttggtgcaaaacttttaccaacaaggtacgtttccaagaatattcccggggcggggagtgaacctttgcatctgaacatgaaactaaatcttgccttcaaggaaggtaaatagaaagtcaggggaaagaaatattgggagtccctgaaaaatcaaaagtgaccagtgaatgcctttagaaaacaagtccttgaatgaatgccaaatgcaggtaaacagtaggacagacagactcccttttgccccatcccagggatacattatttctggtttgttatttccatgcctcttggagttgccccatcccagggatacattatttctggtttgttatttccatgcctcttggagaaggcttagctctgcctgccgctctgttctcatttttggagagacttgtggaagtagtccagcacgccctacgggtgttggcagcaagtgtcctctctcttactttccagacttttccagagtattccccctgctcgggcccagagcattcatgccctctgtcaggaacctacccaggctgctttgcacctgggttgtggcttgcccttgcttctggggtgtacatgtattttttttgtggggtgctaaagactggagactagaataaacaaagtagatgtttttaaatgatgaaggctgatttcagttgtcctcagttattctaagatttctaataaaggtgagaaataagtcttcaagagaaaagaactgttctgttggggccatactgcatgcaccatgggtggtgtctgcgcctgttcattcccacccccgccattgcctcccattccccactaaaagccagtgttgtgggcatttgtcatgtttgtcatccagacccagattaacgtcactgttccctcgactgctaactgcacctccccttccctctgttggacggatggcatccaaaactggaccatgaagaatgtgacctacaaggagaacatcgccaaatgtgagtggagctcagtggattggccactatgacaggtgttgtctgggggtgacctatttatccgtgtgaagtcccagtaagtgaaggaaaggacagtaggagtcaccaagcacctgccctacatcaagcagtgagcaacatgctttcacagcagaggaaactatggcttaggaactgaggtgacaggcctgaggtcccacagcaagggcagaactggaacttgaagtccatttcctttgccagctccacaccacctcagaaaagctcaacagattctagaggattccctctacatgctttgttcagattcctttagtcaataactctaggggccaggcatggtgtctcaaggctataatcccagcactttggggaggccgagatgggctggtcacttgaagccaggagtttgagaccagcctggccaacatggtgaaaccctgtttctactaaaaataatacaaaaaaagtaggcatggtggctcaagcctataattccagccaagattgcaccactacactccagcctgggcaacagggagatagtctgtctcaaaataaaataactttagggagacgacaacccgatttgaaggaagatgtgggacccacatagaacagatgtattcactcaattagtccctactgtacagcaggccccatttcagggcactggggctgtagctcttgaacgggcagtgagcttgcatgatgctctgattattaagaagctgtaatgttttttccaacccagccatccttcgttattctaattactccttacatatacttttgtatatctctgcttctctcttctcaccctcccaccacattaattctgagcactatatcttaatactctaggtatgattaccccaaagaatcaagttaacttaccacctcccacattctaggcactatacattcattcatataaccatgttttgctgggattatctgggggatgcagaaggggctttataattagcaatgccccttactttgacttgcccatcgaatccctcaaagcatacttatcattagtaaaaaaaaaaaattagaccatcgacagcatggtctttagagacagaactgggctgagtctacccctgaacagttgtgtgatttttaaagccatcacttcatcttccagcctcatgcgatcccactgattatgacatagtttcagtggtgccccttgctgcgaaggaggctgagaagggctgtcttgatttggggctgccatctgttaaactaacaaccaggaatctgtttgtaggaaagacaggatctgggggaataaataacaatctgtagccgtggtggctccatgccctcctgacaagattctttgtggtctttcaggccagcatatctttgtgaatttccacctcccggatcttgctgtgggcaccatcttgctcatactctccctgctggtcctctgtggttgcctgatcatgattgtcaagatcctgggctctgtgctcaaggggcaggtcgccactgtcatcaagaagaccatcaacactggtaggtacactgccctcacttgtaggcctcacatgtagtcactgcatggggtgtgggggtcctttagattcccatctagcaatggcctctgcatggagtttctctctcagattggatcagcaccagaagtgaggatgtcattcacctggaaatgttcttggcatcctgggtggggtctagggagcaggcccaagtttccattgcatcttggcaaggtcctgagcaggagttcatatctagagagctgtgagtcaggccttccttcttagcgggtttcctgtatgctcagagctttactggctctgtcaagaccccttagaaaaaggaccccaggtgtctgccctcgcccccacctcccttcactcttgtaaaggaaatgtgatgcataaatacatcagcatctagaggtggccaatgagactgctggctaagaggatttcctgccaagtagggtgtcctggccaatgagagctcgctaagccaatgacatcatcacttcatgaaagctcagtgaaatatagccaagaaaattcgccccccaccccagattgtcttggggaacagagcaactctgggtgcttctgggaaatgcagggcctgatccggtgctagttgggtggaggactcagttggcaggcatgttccaagctgctacagtttcttgatgctctttaaggcagaaatgaaggttgaaggtcaaatgtgggtgctgacaggagaccagggcaggcgaggaagcatgctctcagcactttctggaaggagagtggttacttgggtgggttcactctttctaacctgacctccagggaatctgtgtttttgttttcataacacttacctgtatccttggtttttccatctgaatatgcggagcaaaagacaatggggaatgaaactggattctaaaactctactctgtaatctgagaccatatatgggatgatgtacaacctcacccctaagcccagcccctaccccaggccaccaggccataccttccccggagaggccatgacatctcttccttctgtcttccagatttcccctttccctttgcatggttgactggctacctggccatcctcgtcggggcaggcatgaccttcatcgtacagagcagctctgtgttcacgtcggccttgacccccctgattggtgagttacaccctggcttctccctctggccaccactgccatttcctgtcatcccatggggctgatatgtttgtgttttgtgtttcccccaggaatcggcgtgataaccattgagagggcttatccactcacgctgggctccaacatcggcaccaccaccaccgccatcctggccgccttagccagccctggcaatgcattgaggagttcactccaggtcaggacttggggcacggggacaggggccctgggagtgggaccacccatggtcttgcaaactggtctctaacaagagccaggcttttctctgtactatccaaaatatggaactaatatgtggaggggaagccacgggtaaagttttcaggaccttgatatgagaacaatcaaaactatcagttctgagagaagcagtaagaccctcataactggtggttgtttcagcaaaagtggggtggccccttgatatagatgtggaaaaggtacttaggaagcacagacaccacctcccatctcctcactgcctcctatggggaactttacaattaggagaactccttggcatggaccatctatgttactttgtgcaagacctttgggatttgaatttatttatttatttttattttatttttttgagacagagtctcgcactgtagcccaggctggagtgcagtgatacaatctccactcactgcaacctccacctcccaggttcaagtgattttcctgcctcagcctcccgagcagctgggattacaggcacctgccaccacacctggctgattttttgtatttttagtagagacggggtttccctatgttggccaggctggtcttgaactctggacctcatgatgtgcttgccttggcctcccaaagtgctgggattacaggcgtgagccactgcacccagccggggatttgaattcttatctcaccatttacatactaagtgacctttggcaagtgatctaacctgagcgtcaattccctcatctgaaagatggaggacataacccctatctcattaggattattataaagatctgatgaggcaaagccatctgtgcaacagaaatagaaattcaggcatctaatataaattaaccgcatatgtcatcctaaaattttttagtagatacatttttaaaagtagaaacaggctgattcattcttcaataatgtatttgatctaaaccattatcactttaatatgtcatctgtcaaaaattgagagtctttgaaatctggtgtgtatttcatacttaaagcacatctcagtgtggacatgcggctagtgtattggacagtgctggtccacagcctgggcatggaaattggcaccttgggaaagtgaccaagcgcctggcctctggagccaggctcccaggatgtgaatttccacctcttccatctcccacctgtgggacttgaggagcctccattttctcatgtcaagaaataatggttgccactctgtagagtgttttttgagaattgagataatgcacagttgagtgctcctggccctcccagacacatagtaggtcctcatggaatccaggtaccctctgggctgagccataaggacaaagaaggcctggaaggcccgagactgtgctgcctgtgatgcctgctagcttacctccccctcctcctccctactgccacccgcattgggcaacaggcccctcacctgtccaacctcttgtgttgcagatcgccctgtgccactttttcttcaacatctccggcatcttgctgtggtacccgatcccgttcactcgcctgcccatccgcatggccaaggggctgggcaacatctctgccaagtatcgctggttcgccgtcttctacctgatcatcttcttcttcctgatcccgctgacggtgtttggcctctcgctggccggctggcgggtgctggttggtgtcggggttcccgtcgtcttcatcatcatcctggtactgtgcctccgactcctgcagtctcgctgcccacgcgtcctgccgaagaaactccagaactggaacttcctgccgctgtggatgcgctcgctgaagccctgggatgccgtcgtctccaagttcaccggctgcttccagatgcgctgctgctgctgctgccgcgtgtgctgccgcgcgtgctgcttgctgtgtgactgccccaagtgctgccgctgcagcaagtgctgcgaggacttggaggaggcgcaggaggggcaggatgtccctgtcaaggctcctgagacctttgataacataaccattagcagagaggctcagggtgaggtccctgcctcggactcaaagaccgaatgcacggccttgtaggggacgccccagattgtcagggatggggggatggtccttgagttttgcatgctctcctccctcccacttctgcaccctttcaccacctcgaggagatttgctccccattagcgaatgaaattgatgcagtcctacctaactcgattccctttggcttggtggtaggcctgcagggcacttttattccaacccctggtcactcagtaatcttttactccaggaaggcacaggatggtacctaaagagaattagagaatgaacctggcgggacggatgtctaatcctgcgcctagctgggttggtcagtagaacctattttcagactcaaaaaccatcttcagaaagaaaaggcccagggaaggaatgtatgagaggctctcccagatgaggaagtgtactctctatgactatcaagctcaggcctctccctttttttaaaccaaagtctggcaaccaagagcagcagctccatggcctccttgccccagatcagcctgggtcaggggacatagtgtcattgtttggaaactgcagaccacaaggtgtgggtctatcccacttcctagtgctccccacattccccatcagggcttcctcacgtggacaggtgtgctagtccaggcagttcacttgcagtttccttgtcctcatgcttcggggatgggagccacgcctgaactagagttcaggctggatacatgtgctcacctgctgctcttgtcttcctaagagacagagagtggggcagatggaggagaagaaagtgaggaatgagtagcatagcattctgccaaaagggccccagattcttaatttagcaaactaagaagcccaattcaaaagcattgtggctaaagtctaacgctcctctcttggtcagataacaaaagccctccctgttggatcttttgaaataaaacgtgcaagttatccaggctcgtagcctgcatgctgccaccttgaatcccagggagtatctgcacctggaatagctctccacccctctctgcctccttactttctgtgcaagatgacttcctgggttaacttccttctttccatccacccacccactggaatctctttccaaacatttttccattttcccacagatgggctttgattagctgtcctctctccatgcctgcaaagctccagatttttggggaaagctgtacccaactggactgcccagtgaactgggatcattaagtacagtcgagcacacgtgtgtgcatgggtcaaaggggtgtgttccttctcatcctagatgccttctctgtgccttccacagcctcctgcctgattacaccactgcccccgccccaccctcagccatcccaattcttcctggccagtgcgctccagccttatctaggaaaggaggagtgggtgtagccgtgcagcaagattggggcctcccccatcccagcttctccaccatcccagcaagtcaggatatcagacagtcctcccctgaccctcccccttgtagatatcaattcccaaacagagccaaatactctatatctatagtcacagccctgtacagcatttttcataagttatatagtaaatggtctgcatgatttgtgcttctagtgctctcatttggaaatgaggcaggcttcttctatgaaatgtaaagaaagaaaccactttgtatattttgtaataccacctctgtggccatgcctgccccgcccactctgtatatatgtaagttaaacccgggcaggggctgtggccgtctttgtactctggtgatttttaaaaattgaatctttgtacttgcattgattgtataataattttgagaccaggtctcgctgtgttgctcaggctggtctcaaactcctgagatcaagcaatccgcccacctcagcctcccaaagtgctgagatcacaggcgtgagccaccaccaggcctgattgtaatttttttttttttttttttactggttatgggaagggagaaataaaatcatcaaacccaaaaggagtgtgttgtttttaattacagggaaatagggacctccttggatctattttataaaaatgtgaggtctccttttacctcgttgcactgctaggagcaagatgggtcaccagcagctgtactggagccacccaaaaaaattcggccagggttctcgttcttgtcgtgtctattcaaaccagcacggtctgatccggaaatatggcctcaataagtgccgccaatgtttctgtcagtacgcgaaggatatcggtttcattaagttggactaaatgatcttccttcaaaggattatccaagtcatctactcaatgaaaaaccatgatagttctttgtacataaaataaacatttgaaaaaacaaaacaaaacgaacaaaaaaaaatgtgaggtctcttccttcactgaatgtcactac

SEQ ID NO: 2 - protein, 690 aa, Homo sapiens
MAPWPELGDAQPNPDKYLEGAAGQQPTAPDKSKETNKTDNTEAPVTKIELLPSYSTATLIDEPTEVDDPWNLPTLQDSGIKWSERDTKGKILCFFQGIGRLILLLGFLYFFVCSLDILSSAFQLVGGKMAGQFFSNSSIMSNPLLGLVIGVLVTVLVQSSSTSTSIVVSMVSSSLLTVRAAIPIIMGANIGTSITNTIVALMQVGDRSEFRRAFAGATVHDFFNWLSLLVLLPVEVATHYLEIITQLIVESFHFKNGEDAPDLLKVITKPFTKLIVQLDKKVISQIAMNDEKAKNKSLVKIWCKTFTNKTQINVTVPSTANCTSPSLCWTDGIQNWTMKNVTYKENIAKCQHIFVNFHLPDLAVGTILLILSLLVLCGCLIMIVKILGSVLKGQVATVIKKTINTDFPFPFAWLTGYLAILVGAGMTFIVQSSSVFTSALTPLIGIGVITIERAYPLTLGSNIGTTTTAILAALASPGNALRSSLQIALCHFFFNISGILLWYPIPFTRLPIRMAKGLGNISAKYRWFAVFYLIIFFFLIPLTVFGLSLAGWRVLVGVGVPVVFIIILVLCLRLLQSRCPRVLPKKLQNWNFLPLWMRSLKPWDAVVSKFTGCFQMRCCCCCRVCCRACCLLCDCPKCCRCSKCCEDLEEAQEGQDVPVKAPETFDNITISREAQGEVPASDSKTECTAL

SEQ ID NO: 3 - DNA, 19 bp, Homo sapiens
aagttatcgctttttcatc

SEQ ID NO: 4 - DNA, 1365 bp, Homo sapiens
atgagtggagctcagtggattggccactatgacaggtgttgtctgggggtgacctatttatccgtgtgaagtcccagtaagtgaaggaaaggacagtaggagtcaccaagcacctgccctacatcaagcagtgagcaacatgctttcacagcagaggaaactatggcttaggaactgaggtgacaggcctgaggtcccacagcaagggcagaactggaacttgaagtccatttcctttgccagctccacaccacctcagaaaagctcaacagattctagaggattccctctacatgctttgttcagattcctttagtcaataactctaggggccaggcatggtgtctcaaggctataatcccagcactttggggaggccgagatgggctggtcacttgaagccaggagtttgagaccagcctggccaacatggtgaaaccctgtttctactaaaaataatacaaaaaaagtaggcatggtggctcaagcctataattccagccaagattgcaccactacactccagcctgggcaacagggagatagtctgtctcaaaataaaataactttagggagacgacaacccgatttgaaggaagatgtgggacccacatagaacagatgtattcactcaattagtccctactgtacagcaggccccatttcagggcactggggctgtagctcttgaacgggcagtgagcttgcatgatgctctgattattaagaagctgtaatgttttttccaacccagccatccttcgttattctaattactccttacatatacttttgtatatctctgcttctctcttctcaccctcccaccacattaattctgagcactatatcttaatactctaggtatgattaccccaaagaatcaagttaacttaccacctcccacattctaggcactatacattcattcatataaccatgttttgctgggattatctgggggatgcagaaggggctttataattagcaatgccccttactttgacttgcccatcgaatccctcaaagcatacttatcattagtaaaaaaaaaaaattagaccatcgacagcatggtctttagagacagaactgggctgagtctacccctgaacagttgtgtgatttttaaagccatcacttcatcttccagcctcatgcgatcccactgattatgacatagtttcagtggtgccccttgctgcgaaggaggctgagaagggctgtcttgatttggggctgccatctgttaaactaacaaccaggaatctgtttgtaggaaagacaggatctgggggaataaataacaatctgtagccgtggtggctccatgccctcctgacaagattctttgtggtctttcag

SEQ ID NO: 5 - DNA, 27 bp, Homo sapiens
actatagtttgaagggtactaaggtct

SEQ ID NO: 6 - DNA, 27 bp, Homo sapiens
actattgtttgaaggctactaaggcct

SEQ ID NO: 7 - DNA, 58 bp, Homo sapiens
ctggataaaaaagttatcagccaaattgcaatgaacgatgaaaaagcgaaaaacaaga

SEQ ID NO: 8 - DNA, 62 bp, Homo sapiens
ctggataaaaaagttatcagccaaaaagttatcgctttttcatcaaaaagcgaaaaacaaga

SEQ ID NO: 9 - DNA, 13 bp, Homo sapiens
ccaaatgtgagtg

SEQ ID NO: 10 - DNA, 13 bp, Homo sapiens
ccaaatatgagtg